SEQUENCE LISTING (106 sequences)

SEQ ID NO: 1 - RNA, 19 nt, Artificial sequence
  Description of the artificial sequence sense strand of dsRNA
cagggguccucugugaacu

SEQ ID NO: 2 - RNA, 19 nt, Artificial sequence
  Description of the artificial sequence sense strand of dsRNA
ugcucuucgucaucugacc

SEQ ID NO: 3 - RNA, 19 nt, Artificial sequence
  Description of the artificial sequence sense strand of dsRNA
gcucuucgucaucugacca

SEQ ID NO: 4 - RNA, 19 nt, Artificial sequence
  Description of the artificial sequence sense strand of dsRNA
ggagcuaaaauggcagugc

SEQ ID NO: 5 - RNA, 19 nt, Artificial sequence
  Description of the artificial sequence sense strand of dsRNA
ccugugcagcuggaauucu

SEQ ID NO: 6 - RNA, 19 nt, Artificial sequence
  Description of the artificial sequence sense strand of dsRNA
agaguagcugcaggggucc

SEQ ID NO: 7 - RNA, 19 nt, Artificial sequence
  Description of the artificial sequence sense strand of dsRNA
cugacuauccaguugaugg

SEQ ID NO: 8 - RNA, 19 nt, Artificial sequence
  Description of the artificial sequence sense strand of dsRNA
ccauuccauuguuugugca

SEQ ID NO: 9 - RNA, 19 nt, Artificial sequence
  Description of the artificial sequence sense strand of dsRNA
auaccauuccauuguuugu

SEQ ID NO: 10 - RNA, 19 nt, Artificial sequence
  Description of the artificial sequence sense strand of dsRNA
gcagggguccucugugaac

SEQ ID NO: 11 - RNA, 19 nt, Artificial sequence
  Description of the artificial sequence sense strand of dsRNA
ccaggaccucauggauggg

SEQ ID NO: 12 - RNA, 19 nt, Artificial sequence
  Description of the artificial sequence sense strand of dsRNA
uaccauuccauuguuugug

SEQ ID NO: 13 - RNA, 19 nt, Artificial sequence
  Description of the artificial sequence sense strand of dsRNA
ugugaacuugcucaggaca

SEQ ID NO: 14 - RNA, 19 nt, Artificial sequence
  Description of the artificial sequence sense strand of dsRNA
uggauaucgccaggaugau

SEQ ID NO: 15 - RNA, 19 nt, Artificial sequence
  Description of the artificial sequence sense strand of dsRNA
ugacuauccaguugauggg

SEQ ID NO: 16 - RNA, 19 nt, Artificial sequence
  Description of the artificial sequence sense strand of dsRNA
accaugcagaauacaaaug

SEQ ID NO: 17 - RNA, 19 nt, Artificial sequence
  Description of the artificial sequence sense strand of dsRNA
acuguuggauugauucgaa

SEQ ID NO: 18 - RNA, 19 nt, Artificial sequence
  Description of the artificial sequence sense strand of dsRNA
cuauccaguugaugggcug

SEQ ID NO: 19 - RNA, 19 nt, Artificial sequence
  Description of the artificial sequence sense strand of dsRNA
gacuauccaguugaugggc

SEQ ID NO: 20 - RNA, 19 nt, Artificial sequence
  Description of the artificial sequence sense strand of dsRNA
gcugacuauccaguugaug

SEQ ID NO: 21 - RNA, 19 nt, Artificial sequence
  Description of the artificial sequence sense strand of dsRNA
aauaccauuccauuguuug

SEQ ID NO: 22 - RNA, 19 nt, Artificial sequence
  Description of the artificial sequence sense strand of dsRNA
acccuggugcugacuaucc

SEQ ID NO: 23 - RNA, 19 nt, Artificial sequence
  Description of the artificial sequence sense strand of dsRNA
ugcuuuauucucccauuga

SEQ ID NO: 24 - RNA, 19 nt, Artificial sequence
  Description of the artificial sequence sense strand of dsRNA
aggagcuaaaauggcagug

SEQ ID NO: 25 - RNA, 19 nt, Artificial sequence
  Description of the artificial sequence antisense strand of dsRNA
aguucacagaggaccccug

SEQ ID NO: 26 - RNA, 19 nt, Artificial sequence
  Description of the artificial sequence antisense strand of dsRNA
ggucagaugacgaagagca

SEQ ID NO: 27 - RNA, 19 nt, Artificial sequence
  Description of the artificial sequence antisense strand of dsRNA
uggucagaugacgaagagc

SEQ ID NO: 28 - RNA, 19 nt, Artificial sequence
  Description of the artificial sequence antisense strand of dsRNA
gcacugccauuuuagcucc

SEQ ID NO: 29 - RNA, 19 nt, Artificial sequence
  Description of the artificial sequence antisense strand of dsRNA
agaauuccagcugcacagg

SEQ ID NO: 30 - RNA, 19 nt, Artificial sequence
  Description of the artificial sequence antisense strand of dsRNA
ggaccccugcagcuacucu

SEQ ID NO: 31 - RNA, 19 nt, Artificial sequence
  Description of the artificial sequence antisense strand of dsRNA
ccaucaacuggauagucag

SEQ ID NO: 32 - RNA, 19 nt, Artificial sequence
  Description of the artificial sequence antisense strand of dsRNA
ugcacaaacaauggaaugg

SEQ ID NO: 33 - RNA, 19 nt, Artificial sequence
  Description of the artificial sequence antisense strand of dsRNA
acaaacaauggaaugguau

SEQ ID NO: 34 - RNA, 19 nt, Artificial sequence
  Description of the artificial sequence antisense strand of dsRNA
guucacagaggaccccugc

SEQ ID NO: 35 - RNA, 19 nt, Artificial sequence
  Description of the artificial sequence antisense strand of dsRNA
cccauccaugagguccugg

SEQ ID NO: 36 - RNA, 19 nt, Artificial sequence
  Description of the artificial sequence antisense strand of dsRNA
cacaaacaauggaauggua

SEQ ID NO: 37 - RNA, 19 nt, Artificial sequence
  Description of the artificial sequence antisense strand of dsRNA
uguccugagcaaguucaca

SEQ ID NO: 38 - RNA, 19 nt, Artificial sequence
  Description of the artificial sequence antisense strand of dsRNA
aucauccuggcgauaucca

SEQ ID NO: 39 - RNA, 19 nt, Artificial sequence
  Description of the artificial sequence antisense strand of dsRNA
cccaucaacuggauaguca

SEQ ID NO: 40 - RNA, 19 nt, Artificial sequence
  Description of the artificial sequence antisense strand of dsRNA
cauuuguauucugcauggu

SEQ ID NO: 41 - RNA, 19 nt, Artificial sequence
  Description of the artificial sequence antisense strand of dsRNA
uucgaaucaauccaacagu

SEQ ID NO: 42 - RNA, 19 nt, Artificial sequence
  Description of the artificial sequence antisense strand of dsRNA
cagcccaucaacuggauag

SEQ ID NO: 43 - RNA, 19 nt, Artificial sequence
  Description of the artificial sequence antisense strand of dsRNA
gcccaucaacuggauaguc

SEQ ID NO: 44 - RNA, 19 nt, Artificial sequence
  Description of the artificial sequence antisense strand of dsRNA
caucaacuggauagucagc

SEQ ID NO: 45 - RNA, 19 nt, Artificial sequence
  Description of the artificial sequence antisense strand of dsRNA
caaacaauggaaugguauu

SEQ ID NO: 46 - RNA, 19 nt, Artificial sequence
  Description of the artificial sequence antisense strand of dsRNA
ggauagucagcaccagggu

SEQ ID NO: 47 - RNA, 19 nt, Artificial sequence
  Description of the artificial sequence antisense strand of dsRNA
ucaaugggagaauaaagca

SEQ ID NO: 48 - RNA, 19 nt, Artificial sequence
  Description of the artificial sequence antisense strand of dsRNA
cacugccauuuuagcuccu

SEQ ID NO: 49 - DNA, 21 bp, Artificial sequence
  Description of the artificial sequence sense strand of dsRNA
cagggguccucugugaacutt

SEQ ID NO: 50 - DNA, 21 bp, Artificial sequence
  Description of the artificial sequence sense strand of dsRNA
ugcucuucgucaucugacctt

SEQ ID NO: 51 - DNA, 21 bp, Artificial sequence
  Description of the artificial sequence sense strand of dsRNA
gcucuucgucaucugaccatt

SEQ ID NO: 52 - DNA, 21 bp, Artificial sequence
  Description of the artificial sequence sense strand of dsRNA
ggagcuaaaauggcagugctt

SEQ ID NO: 53 - DNA, 21 bp, Artificial sequence
  Description of the artificial sequence sense strand of dsRNA
ccugugcagcuggaauucutt

SEQ ID NO: 54 - DNA, 21 bp, Artificial sequence
  Description of the artificial sequence sense strand of dsRNA
agaguagcugcaggggucctt

SEQ ID NO: 55 - DNA, 21 bp, Artificial sequence
  Description of the artificial sequence sense strand of dsRNA
cugacuauccaguugauggtt

SEQ ID NO: 56 - DNA, 21 bp, Artificial sequence
  Description of the artificial sequence sense strand of dsRNA
ccauuccauuguuugugcatt

SEQ ID NO: 57 - DNA, 21 bp, Artificial sequence
  Description of the artificial sequence sense strand of dsRNA
auaccauuccauuguuugutt

SEQ ID NO: 58 - DNA, 21 bp, Artificial sequence
  Description of the artificial sequence sense strand of dsRNA
gcagggguccucugugaactt

SEQ ID NO: 59 - DNA, 21 bp, Artificial sequence
  Description of the artificial sequence sense strand of dsRNA
ccaggaccucauggaugggtt

SEQ ID NO: 60 - DNA, 21 bp, Artificial sequence
  Description of the artificial sequence sense strand of dsRNA
uaccauuccauuguuugugtt

SEQ ID NO: 61 - DNA, 21 bp, Artificial sequence
  Description of the artificial sequence sense strand of dsRNA
ugugaacuugcucaggacatt

SEQ ID NO: 62 - DNA, 21 bp, Artificial sequence
  Description of the artificial sequence sense strand of dsRNA
uggauaucgccaggaugautt

SEQ ID NO: 63 - DNA, 21 bp, Artificial sequence
  Description of the artificial sequence sense strand of dsRNA
ugacuauccaguugaugggtt

SEQ ID NO: 64 - DNA, 21 bp, Artificial sequence
  Description of the artificial sequence sense strand of dsRNA
accaugcagaauacaaaugtt

SEQ ID NO: 65 - DNA, 21 bp, Artificial sequence
  Description of the artificial sequence sense strand of dsRNA
acuguuggauugauucgaatt

SEQ ID NO: 66 - DNA, 21 bp, Artificial sequence
  Description of the artificial sequence sense strand of dsRNA
cuauccaguugaugggcugtt

SEQ ID NO: 67 - DNA, 21 bp, Artificial sequence
  Description of the artificial sequence sense strand of dsRNA
gacuauccaguugaugggctt

SEQ ID NO: 68 - DNA, 21 bp, Artificial sequence
  Description of the artificial sequence sense strand of dsRNA
gcugacuauccaguugaugtt

SEQ ID NO: 69 - DNA, 21 bp, Artificial sequence
  Description of the artificial sequence sense strand of dsRNA
aauaccauuccauuguuugtt

SEQ ID NO: 70 - DNA, 21 bp, Artificial sequence
  Description of the artificial sequence sense strand of dsRNA
acccuggugcugacuaucctt

SEQ ID NO: 71 - DNA, 21 bp, Artificial sequence
  Description of the artificial sequence sense strand of dsRNA
ugcuuuauucucccauugatt

SEQ ID NO: 72 - DNA, 21 bp, Artificial sequence
  Description of the artificial sequence sense strand of dsRNA
aggagcuaaaauggcagugtt

SEQ ID NO: 73 - DNA, 21 bp, Artificial sequence
  Description of the artificial sequence antisense strand of dsRNA
aguucacagaggaccccugtt

SEQ ID NO: 74 - DNA, 21 bp, Artificial sequence
  Description of the artificial sequence antisense strand of dsRNA
ggucagaugacgaagagcatt

SEQ ID NO: 75 - DNA, 21 bp, Artificial sequence
  Description of the artificial sequence antisense strand of dsRNA
uggucagaugacgaagagctt

SEQ ID NO: 76 - DNA, 21 bp, Artificial sequence
  Description of the artificial sequence antisense strand of dsRNA
gcacugccauuuuagcucctt

SEQ ID NO: 77 - DNA, 21 bp, Artificial sequence
  Description of the artificial sequence antisense strand of dsRNA
agaauuccagcugcacaggtt

SEQ ID NO: 78 - DNA, 21 bp, Artificial sequence
  Description of the artificial sequence antisense strand of dsRNA
ggaccccugcagcuacucutt

SEQ ID NO: 79 - DNA, 21 bp, Artificial sequence
  Description of the artificial sequence antisense strand of dsRNA
ccaucaacuggauagucagtt

SEQ ID NO: 80 - DNA, 21 bp, Artificial sequence
  Description of the artificial sequence antisense strand of dsRNA
ugcacaaacaauggaauggtt

SEQ ID NO: 81 - DNA, 21 bp, Artificial sequence
  Description of the artificial sequence antisense strand of dsRNA
acaaacaauggaaugguautt

SEQ ID NO: 82 - DNA, 21 bp, Artificial sequence
  Description of the artificial sequence antisense strand of dsRNA
guucacagaggaccccugctt

SEQ ID NO: 83 - DNA, 21 bp, Artificial sequence
  Description of the artificial sequence antisense strand of dsRNA
cccauccaugagguccuggtt

SEQ ID NO: 84 - DNA, 21 bp, Artificial sequence
  Description of the artificial sequence antisense strand of dsRNA
cacaaacaauggaaugguatt

SEQ ID NO: 85 - DNA, 21 bp, Artificial sequence
  Description of the artificial sequence antisense strand of dsRNA
uguccugagcaaguucacatt

SEQ ID NO: 86 - DNA, 21 bp, Artificial sequence
  Description of the artificial sequence antisense strand of dsRNA
aucauccuggcgauauccatt

SEQ ID NO: 87 - DNA, 21 bp, Artificial sequence
  Description of the artificial sequence antisense strand of dsRNA
cccaucaacuggauagucatt

SEQ ID NO: 88 - DNA, 21 bp, Artificial sequence
  Description of the artificial sequence antisense strand of dsRNA
cauuuguauucugcauggutt

SEQ ID NO: 89 - DNA, 21 bp, Artificial sequence
  Description of the artificial sequence antisense strand of dsRNA
uucgaaucaauccaacagutt

SEQ ID NO: 90 - DNA, 21 bp, Artificial sequence
  Description of the artificial sequence antisense strand of dsRNA
cagcccaucaacuggauagtt

SEQ ID NO: 91 - DNA, 21 bp, Artificial sequence
  Description of the artificial sequence antisense strand of dsRNA
gcccaucaacuggauaguctt

SEQ ID NO: 92 - DNA, 21 bp, Artificial sequence
  Description of the artificial sequence antisense strand of dsRNA
caucaacuggauagucagctt

SEQ ID NO: 93 - DNA, 21 bp, Artificial sequence
  Description of the artificial sequence antisense strand of dsRNA
caaacaauggaaugguauutt

SEQ ID NO: 94 - DNA, 21 bp, Artificial sequence
  Description of the artificial sequence antisense strand of dsRNA
ggauagucagcaccagggutt

SEQ ID NO: 95 - DNA, 21 bp, Artificial sequence
  Description of the artificial sequence antisense strand of dsRNA
ucaaugggagaauaaagcatt

SEQ ID NO: 96 - DNA, 21 bp, Artificial sequence
  Description of the artificial sequence antisense strand of dsRNA
cacugccauuuuagcuccutt

SEQ ID NO: 97 - DNA, 3720 bp, Homo sapiens
  cDNA of human CTNNB1
aggauacagcggcuucugcgcgacuuauaagagcuccuugugcggcgccauuuuaagccucucggucuguggcagcagcguuggcccggccccgggagcggagagcgaggggaggcggagacggaggaaggucugaggagcagcuucaguccccgccgagccgccaccgcaggucgaggacggucggacucccgcggcgggaggagccuguuccccugaggguauuugaaguauaccauacaacuguuuugaaaauccagcguggacaauggcuacucaagcugauuugauggaguuggacauggccauggaaccagacagaaaagcggcuguuagucacuggcagcaacagucuuaccuggacucuggaauccauucuggugccacuaccacagcuccuucucugagugguaaaggcaauccugaggaagaggauguggauaccucccaaguccuguaugagugggaacagggauuuucucaguccuucacucaagaacaaguagcugauauugauggacaguaugcaaugacucgagcucagaggguacgagcugcuauguucccugagacauuagaugagggcaugcagaucccaucuacacaguuugaugcugcucaucccacuaauguccagcguuuggcugaaccaucacagaugcugaaacaugcaguuguaaacuugauuaacuaucaagaugaugcagaacuugccacacgugcaaucccugaacugacaaaacugcuaaaugacgaggaccagguggugguuaauaaggcugcaguuaugguccaucagcuuucuaaaaaggaagcuuccagacacgcuaucaugcguucuccucagauggugucugcuauuguacguaccaugcagaauacaaaugauguagaaacagcucguuguaccgcugggaccuugcauaaccuuucccaucaucgugagggcuuacuggccaucuuuaagucuggaggcauuccugcccuggugaaaaugcuugguucaccaguggauucuguguuguuuuaugccauuacaacucuccacaaccuuuuauuacaucaagaaggagcuaaaauggcagugcguuuagcuggugggcugcagaaaaugguugccuugcucaacaaaacaaauguuaaauucuuggcuauuacgacagacugccuucaaauuuuagcuuauggcaaccaagaaagcaagcucaucauacuggcuagugguggaccccaagcuuuaguaaauauaaugaggaccuauacuuacgaaaaacuacuguggaccacaagcagagugcugaaggugcuaucugucugcucuaguaauaagccggcuauuguagaagcugguggaaugcaagcuuuaggacuucaccugacagauccaagucaacgucuuguucagaacugucuuuggacucucaggaaucuuucagaugcugcaacuaaacaggaagggauggaaggucuccuugggacucuuguucagcuucuggguucagaugauauaaauguggucaccugugcagcuggaauucuuucuaaccucacuugcaauaauuauaagaacaagaugauggucugccaagugggugguauagaggcucuugugcguacuguccuucgggcuggugacagggaagacaucacugagccugccaucugugcucuucgucaucugaccagccgacaccaagaagcagagauggcccagaaugcaguucgccuucacuauggacuaccaguugugguuaagcucuuacacccaccaucccacuggccucugauaaaggcuacuguuggauugauucgaaaucuugcccuuugucccgcaaaucaugcaccuuugcgugagcagggugccauuccacgacuaguucaguugcuuguucgugcacaucaggauacccagcgccguacguccaugggugggacacagcagcaauuuguggaggggguccgcauggaagaaauaguugaagguuguaccggagcccuucacauccuagcucgggauguucacaaccgaauuguuaucagaggacuaaauaccauuccauuguuugugcagcugcuuuauucucccauugaaaacauccaaagaguagcugcagggguccucugugaacuugcucaggacaaggaagcugcagaagcuauugaagcugagggagccacagcuccucugacagaguuacuucacucuaggaaugaagguguggcgacauaugcagcugcuguuuuguuccgaaugucugaggacaagccacaagauuacaagaaacggcuuucaguugagcugaccagcucucucuucagaacagagccaauggcuuggaaugagacugcugaucuuggacuugauauuggugcccagggagaaccccuuggauaucgccaggaugauccuagcuaucguucuuuucacucugguggauauggccaggaugccuuggguauggaccccaugauggaacaugagauggguggccaccacccuggugcugacuauccaguugaugggcugccagaucuggggcaugcccaggaccucauggaugggcugccuccaggugacagcaaucagcuggccugguuugauacugaccuguaaaucauccuuuagguaagaaguuuuaaaaagccaguuuggguaaaauacuuuuacucugccuacagaacuucagaaagacuugguugguagggugggagugguuuaggcuauuuguaaaucugccacaaaaacagguauauacuuugaaaggagaugucuuggaacauuggaauguucucagauuucugguuguuaugugaucauguguggaaguuauuaacuuuaauguuuuuugccacagcuuuugcaacuuaauacucaaaugaguaacauuugcuguuuuaaacauuaauagcagccuuucucucuuuauacagcuguauugucugaacuugcauugugauuggccuguagaguugcugagagggcucgaggggugggcugguaucucagaaagugccugacacacuaaccaagcugaguuuccuaugggaacaauugaaguaaacuuuuuguucugguccuuuuuggucgaggaguaacaauacaaauggauuuugggagugacucaagaagugaagaaugcacaagaauggaucacaagauggaauuuaucaaacccuagccuugcuuguuaaauuuuuuuuuuuuuuuuuuuaagaauaucuguaaugguacugacuuugcuugcuuugaaguagcucuuuuuuuuuuuuuuuuuuuuuuuuugcaguaacuguuuuuuaagucucucguaguguuaaguuauagugaauacugcuacagcaauuucuaauuuuuaagaauugaguaaugguguagaacacuaauucauaaucacucuaauuaauuguaaucugaauaaaguguaacaauuguguagccuuuuuguauaaaauagacaaauagaaaaugguccaauuaguuuccuuuuuaauaugcuuaaaauaagcagguggaucuauuucauguuuuugaucaaaaacuauuugggauauguauggguaggguaaaucaguaagagguguuauuuggaaccuuguuuuggacaguuuaccaguugccuuuuaucccaaaguuguuguaaccugcugugauacgaugcuucaagagaaaaugcgguuauaaaaaaugguucagaauuaaacuuuuaauucauucgauug

SEQ ID NO: 98 - DNA, 3415 bp, Homo sapiens
  cDNA of human CTNNB1
aggauacagcggcuucugcgcgacuuauaagagcuccuugugcggcgccauuuuaagccucucggucuguggcagcagcguuggcccggccccgggagcggagagcgaggggaggcggagacggaggaaggucugaggagcagcuucaguccccgccgagccgccaccgcaggucgaggacggucggacucccgcggcgggaggagccuguuccccugaggguauuugaaguauaccauacaacuguuuugaaaauccagcguggacaauggcuacucaagcugauuugauggaguuggacauggccauggaaccagacagaaaagcggcuguuagucacuggcagcaacagucuuaccuggacucuggaauccauucuggugccacuaccacagcuccuucucugagugguaaaggcaauccugaggaagaggauguggauaccucccaaguccuguaugagugggaacagggauuuucucaguccuucacucaagaacaaguagcugauauugauggacaguaugcaaugacucgagcucagaggguacgagcugcuauguucccugagacauuagaugagggcaugcagaucccaucuacacaguuugaugcugcucaucccacuaauguccagcguuuggcugaaccaucacagaugcugaaacaugcaguuguaaacuugauuaacuaucaagaugaugcagaacuugccacacgugcaaucccugaacugacaaaacugcuaaaugacgaggaccagguggugguuaauaaggcugcaguuaugguccaucagcuuucuaaaaaggaagcuuccagacacgcuaucaugcguucuccucagauggugucugcuauuguacguaccaugcagaauacaaaugauguagaaacagcucguuguaccgcugggaccuugcauaaccuuucccaucaucgugagggcuuacuggccaucuuuaagucuggaggcauuccugcccuggugaaaaugcuugguucaccaguggauucuguguuguuuuaugccauuacaacucuccacaaccuuuuauuacaucaagaaggagcuaaaauggcagugcguuuagcuggugggcugcagaaaaugguugccuugcucaacaaaacaaauguuaaauucuuggcuauuacgacagacugccuucaaauuuuagcuuauggcaaccaagaaagcaagcucaucauacuggcuagugguggaccccaagcuuuaguaaauauaaugaggaccuauacuuacgaaaaacuacuguggaccacaagcagagugcugaaggugcuaucugucugcucuaguaauaagccggcuauuguagaagcugguggaaugcaagcuuuaggacuucaccugacagauccaagucaacgucuuguucagaacugucuuuggacucucaggaaucuuucagaugcugcaacuaaacaggaagggauggaaggucuccuugggacucuuguucagcuucuggguucagaugauauaaauguggucaccugugcagcuggaauucuuucuaaccucacuugcaauaauuauaagaacaagaugauggucugccaagugggugguauagaggcucuugugcguacuguccuucgggcuggugacagggaagacaucacugagccugccaucugugcucuucgucaucugaccagccgacaccaagaagcagagauggcccagaaugcaguucgccuucacuauggacuaccaguugugguuaagcucuuacacccaccaucccacuggccucugauaaaggcuacuguuggauugauucgaaaucuugcccuuugucccgcaaaucaugcaccuuugcgugagcagggugccauuccacgacuaguucaguugcuuguucgugcacaucaggauacccagcgccguacguccaugggugggacacagcagcaauuuguggaggggguccgcauggaagaaauaguugaagguuguaccggagcccuucacauccuagcucgggauguucacaaccgaauuguuaucagaggacuaaauaccauuccauuguuugugcagcugcuuuauucucccauugaaaacauccaaagaguagcugcagggguccucugugaacuugcucaggacaaggaagcugcagaagcuauugaagcugagggagccacagcuccucugacagaguuacuucacucuaggaaugaagguguggcgacauaugcagcugcuguuuuguuccgaaugucugaggacaagccacaagauuacaagaaacggcuuucaguugagcugaccagcucucucuucagaacagagccaauggcuuggaaugagacugcugaucuuggacuugauauuggugcccagggagaaccccuuggauaucgccaggaugauccuagcuaucguucuuuucacucugguggauauggccaggaugccuuggguauggaccccaugauggaacaugagauggguggccaccacccuggugcugacuauccaguugaugggcugccagaucuggggcaugcccaggaccucauggaugggcugccuccaggugacagcaaucagcuggccugguuugauacugaccuguaaaucauccuuuagcuguauugucugaacuugcauugugauuggccuguagaguugcugagagggcucgaggggugggcugguaucucagaaagugccugacacacuaaccaagcugaguuuccuaugggaacaauugaaguaaacuuuuuguucugguccuuuuuggucgaggaguaacaauacaaauggauuuugggagugacucaagaagugaagaaugcacaagaauggaucacaagauggaauuuaucaaacccuagccuugcuuguuaaauuuuuuuuuuuuuuuuuuuaagaauaucuguaaugguacugacuuugcuugcuuugaaguagcucuuuuuuuuuuuuuuuuuuuuuuuuugcaguaacuguuuuuuaagucucucguaguguuaaguuauagugaauacugcuacagcaauuucuaauuuuuaagaauugaguaaugguguagaacacuaauucauaaucacucuaauuaauuguaaucugaauaaaguguaacaauuguguagccuuuuuguauaaaauagacaaauagaaaaugguccaauuaguuuccuuuuuaauaugcuuaaaauaagcagguggaucuauuucauguuuuugaucaaaaacuauuugggauauguauggguaggguaaaucaguaagagguguuauuuggaaccuuguuuuggacaguuuaccaguugccuuuuaucccaaaguuguuguaaccugcugugauacgaugcuucaagagaaaaugcgguuauaaaaaaugguucagaauuaaacuuuuaauucauucgauug

SEQ ID NO: 99 - DNA, 3256 bp, Homo sapiens
  cDNA of human CTNNB1
aggauacagcggcuucugcgcgacuuauaagagcuccuugugcggcgccauuuuaagccucucggucuguggcagcagcguuggcccggccccgggagcggagagcgaggggaggcggagacggaggaaggucugaggagcagcuucaguccccgccgagccgccaccgcaggucgaggacggucggacucccgcggcgggaggagccuguuccccugaggguauuugaaguauaccauacaacuguuuugaaaauccagcguggacaauggcuacucaagcugauuugauggaguuggacauggccauggaaccagacagaaaagcggcuguuagucacuggcagcaacagucuuaccuggacucuggaauccauucuggugccacuaccacagcuccuucucugagugguaaaggcaauccugaggaagaggauguggauaccucccaaguccuguaugagugggaacagggauuuucucaguccuucacucaagaacaaguagcugauauugauggacaguaugcaaugacucgagcucagaggguacgagcugcuauguucccugagacauuagaugagggcaugcagaucccaucuacacaguuugaugcugcucaucccacuaauguccagcguuuggcugaaccaucacagaugcugaaacaugcaguuguaaacuugauuaacuaucaagaugaugcagaacuugccacacgugcaaucccugaacugacaaaacugcuaaaugacgaggaccagguggugguuaauaaggcugcaguuaugguccaucagcuuucuaaaaaggaagcuuccagacacgcuaucaugcguucuccucagauggugucugcuauuguacguaccaugcagaauacaaaugauguagaaacagcucguuguaccgcugggaccuugcauaaccuuucccaucaucgugagggcuuacuggccaucuuuaagucuggaggcauuccugcccuggugaaaaugcuugguucaccaguggauucuguguuguuuuaugccauuacaacucuccacaaccuuuuauuacaucaagaaggagcuaaaauggcagugcguuuagcuggugggcugcagaaaaugguugccuugcucaacaaaacaaauguuaaauucuuggcuauuacgacagacugccuucaaauuuuagcuuauggcaaccaagaaagcaagcucaucauacuggcuagugguggaccccaagcuuuaguaaauauaaugaggaccuauacuuacgaaaaacuacuguggaccacaagcagagugcugaaggugcuaucugucugcucuaguaauaagccggcuauuguagaagcugguggaaugcaagcuuuaggacuucaccugacagauccaagucaacgucuuguucagaacugucuuuggacucucaggaaucuuucagaugcugcaacuaaacaggaagggauggaaggucuccuugggacucuuguucagcuucuggguucagaugauauaaauguggucaccugugcagcuggaauucuuucuaaccucacuugcaauaauuauaagaacaagaugauggucugccaagugggugguauagaggcucuugugcguacuguccuucgggcuggugacagggaagacaucacugagccugccaucugugcucuucgucaucugaccagccgacaccaagaagcagagauggcccagaaugcaguucgccuucacuauggacuaccaguugugguuaagcucuuacacccaccaucccacuggccucugauaaaggcuacuguuggauugauucgaaaucuugcccuuugucccgcaaaucaugcaccuuugcgugagcagggugccauuccacgacuaguucaguugcuuguucgugcacaucaggauacccagcgccguacguccaugggugggacacagcagcaauuuguggaggggguccgcauggaagaaauaguugaagguuguaccggagcccuucacauccuagcucgggauguucacaaccgaauuguuaucagaggacuaaauaccauuccauuguuugugcagcugcuuuauucucccauugaaaacauccaaagaguagcugcagggguccucugugaacuugcucaggacaaggaagcugcagaagcuauugaagcugagggagccacagcuccucugacagaguuacuucacucuaggaaugaagguguggcgacauaugcagcugcuguuuuguuccgaaugucugaggacaagccacaagauuacaagaaacggcuuucaguugagcugaccagcucucucuucagaacagagccaauggcuuggaaugagacugcugaucuuggacuugauauuggugcccagggagaaccccuuggauaucgccaggaugauccuagcuaucguucuuuucacucugguggauauggccaggaugccuuggguauggaccccaugauggaacaugagauggguggccaccacccuggugcugacuauccaguugaugggcugccagaucuggggcaugcccaggaccucauggaugggcugccuccaggugacagcaaucagcuggccugguuugauacugaccuguaaaucauccuuuaggaguaacaauacaaauggauuuugggagugacucaagaagugaagaaugcacaagaauggaucacaagauggaauuuaucaaacccuagccuugcuuguuaaauuuuuuuuuuuuuuuuuuuaagaauaucuguaaugguacugacuuugcuugcuuugaaguagcucuuuuuuuuuuuuuuuuuuuuuuuuugcaguaacuguuuuuuaagucucucguaguguuaaguuauagugaauacugcuacagcaauuucuaauuuuuaagaauugaguaaugguguagaacacuaauucauaaucacucuaauuaauuguaaucugaauaaaguguaacaauuguguagccuuuuuguauaaaauagacaaauagaaaaugguccaauuaguuuccuuuuuaauaugcuuaaaauaagcagguggaucuauuucauguuuuugaucaaaaacuauuugggauauguauggguaggguaaaucaguaagagguguuauuuggaaccuuguuuuggacaguuuaccaguugccuuuuaucccaaaguuguuguaaccugcugugauacgaugcuucaagagaaaaugcgguuauaaaaaaugguucagaauuaaacuuuuaauucauucgauug

SEQ ID NO: 100 - DNA, 3464 bp, Macaca mulatta
  cDNA of rhesus monkey CTNNB1
gagcgaggggaggcggagacggaggaagguccgaggagcagcuucaguccucgccgagccgccaccgcaggucgaggacggucggacucccgcgacgggaggagccuguuccccugaggguauuugaaguauaccauacaacuguuuugaaaauccagcguggacaauggcuacucaagaacaaguagcugauauugauggacaguaugcaaugacucgagcucagaggguacgagcugcuauguucccugagacauuagaugagggcaugcagaucccaucuacacaguuugaugcugcucaucccacuaauguccagcguuuggcugaaccaucacagaugcugaaacaugcaguuguaaacuugauuaacuaucaagaugaugcagaacuugccacacgugcaaucccugaacugacaaaacugcuaaaugaugaggaccagguggugguuaauaaggcugcaguuaugguccaucagcuuucuaaaaaggaagcuuccagacacgcuaucaugcguucuccucagauggugucugcuauuguacguaccaugcagaauacaaaugauguagaaacagcucguuguaccgcugggaccuugcauaaccuuucccaucaucgggagggcuuguuggccaucuuuaagucuggaggcauuccugcccuggugaaaaugcuugguucaccaguggauucuguguuguuuuaugccauuacaacucuccacaaccuuuuauuacaucaagaaggagcuaaaauggcagugcguuuagcuggcgggcuacagaaaaugguugccuugcucaacaaaacaaacguuaaauucuuggcuauuacgacagacugccuucaaauuuuagcauauggcaaccaagaaagcaagcugaucauacuggcuagugguggaccccaagcuuuaguaaauauaaugaggaccuauacuuaugagaaacuacuguggaccacaagcagagugcugaaggugcuauccgucugcucuaguaauaagccagcuauuguagaagcugguggaaugcaagcuuuaggacuucaccugacagauccaagucaacgucuuguucagaacugucuuuggacucucaggaaucuuucagaugcugcaacuaaacaggaagggauggaaggucuccuugggacucuuguucagcuucuggguucagaugauauaaauguggucaccugugcagcuggaauucuuucuaaccucacuugcaauaauuauaagaauaagaugauggucugccaagugggugguauagaggcucuugugcguacuguccuucgggcuggugacagggaagacaucacugagccugccaucugugcucuucgucaucugaccagccgacaccaagaagcagagauggcccagaaugcaguucgccuucacuauggacuaccaguugugguuaagcucuuacacccaccaucccacuggccucugauaaaggcuacuguuggauugauucgaaaucuugcccuuuguccagcaaaucaugcaccuuugcgugagcagggugccauuccacgacuaguucaguugcuuguucgugcacaucaggauacccagcgccguacguccaugggugggacacagcagcaauuuguggaggggguccgcauggaagaaauaguugaagguuguacuggagcccuucacauccuagcucgggauguucacaaccgaauuguaaucagaggacuaaauaccauuccauuguuugugcagcugcuuuauucucccauugaaaacauccaaagaguagcugcagggguccucugugaacuugcucaggacaaggaagcugcagaagcgauugaagcugagggagccacagcuccucugacagaguuacuucacucuaggaaugaagguguggcgacguaugcagcugcuguuuuguuccgaaugucugaggacaagccacaagauuacaagaaacggcuuucaguugagcugaccagcucucucuucagaacggagccaauggcuuggaaugagacugcggaucuuggacuugauauuggugcccagggagaaccccuuggauaucgccaggaugauccuagcuaucguucuuuucacucugguggauauggccaggaugccuuggguauggaccccaugauggaacaugagauggguggccaccacccuggugcugacuauccaguugaugggcugccagaucugggacaugcccaggaccucauggaugggcugccuccaggugauagcaaucagcuggccugguuugauacugaccuguaaaucauccuuuagguaagaaguuuuaaaaagccaguuuggguaaaauacuuuuacucugccuacagaauuucagaaagacuugguugguagggugggagugauuuaggcuauuuguaaaucugccacaaaaacagguauauacuuugaaaggagaugucuuggaacaucggaauguucucagauuucugguuguuaugugaucauguguggaaguuauuaacuuuaauguuuuuugccacagcuuuugcaacuuaauacucaaaugaguaacauuugcuguuuuaaacauuaauagcagccuuucucucuuuauacagcuguauugucugaacuugcauugugauuggccuguagaguugcugagagggcucgaggggugggcugguaucucagaaagugccugacacacuaaccaagcugaguuuccuaugggaacaauugaaguaaacuuuuuguucugguccuuuuuggucgaggaguaacaauacaaauggauuuugggagugacucaagaagugaagaaugcacaagaauggaucacaagauggaauuuaucaaacccuagccuugcuuguuaaauuuuuuuuuuuuuuaauaucuguaaugguacugacuuugcuugcuuugaaguagcucuuuaauuuuuuuuuuuuuuuuuuugcaguaacuguuaguuuaagucucucguaguguuaaguuauagugaauacugcuacagcaauuucuaauuuuuaagaauugaguaaugguguagaacacuaauucauaaucacucuaauaauuguaaucugaauaaaguguaacauuguguagccuuuuuguauaaaauagacaaauagaaaaugguccaauuaguuuccuuuuuaauaugcuuaaaauaagcagguggaucuauuucauguuuuugaucaaaaacuuuauuugggauauguauggguaggguaaaucaguaagagguguuauuuggaaccuuguuuuggacaguuuaccaguugccuuuuaucccaaaguuguuguaaccugcugugauacaaugcuucaagagaaaaugcgguuauaaaaaaugguucagaauuaaacuuuuaauucauucgauugugucacucuuuuuuuuucuugucauuaaauggugagggugguaaguuuauccaacucaacuuuauacuga

SEQ ID NO: 101 - DNA, 3671 bp, Macaca mulatta
  cDNA of rhesus monkey CTNNB1
ggagccaaacuucgucgcaggcgcgcgguccgggcggcgggcugggagccgggcugggcgcagccgggaggccugggguugggaguggggggcucagagcagguacaaggguauuugaaguauaccauacaacuguuuugaaaauccagcguggacaauggcuacucaagcugauuugauggaguuggacauggccauggaaccagacagaaaagcggcuguuagucacuggcagcaacagucuuaccuggacucuggaauccauucuggugccacuaccacagcuccuucucugagugguaaaggcaauccugaggaagaggauguggauaccucccaaguccuguaugagugggaacagggauuuucucaguccuucacucaagaacaaguagcugauauugauggacaguaugcaaugacucgagcucagaggguacgagcugcuauguucccugagacauuagaugagggcaugcagaucccaucuacacaguuugaugcugcucaucccacuaauguccagcguuuggcugaaccaucacagaugcugaaacaugcaguuguaaacuugauuaacuaucaagaugaugcagaacuugccacacgugcaaucccugaacugacaaaacugcuaaaugaugaggaccagguggugguuaauaaggcugcaguuaugguccaucagcuuucuaaaaaggaagcuuccagacacgcuaucaugcguucuccucagauggugucugcuauuguacguaccaugcagaauacaaaugauguagaaacagcucguuguaccgcugggaccuugcauaaccuuucccaucaucgggagggcuuguuggccaucuuuaagucuggaggcauuccugcccuggugaaaaugcuugguucaccaguggauucuguguuguuuuaugccauuacaacucuccacaaccuuuuauuacaucaagaaggagcuaaaauggcagugcguuuagcuggcgggcuacagaaaaugguugccuugcucaacaaaacaaacguuaaauucuuggcuauuacgacagacugccuucaaauuuuagcauauggcaaccaagaaagcaagcugaucauacuggcuagugguggaccccaagcuuuaguaaauauaaugaggaccuauacuuaugagaaacuacuguggaccacaagcagagugcugaaggugcuauccgucugcucuaguaauaagccagcuauuguagaagcugguggaaugcaagcuuuaggacuucaccugacagauccaagucaacgucuuguucagaacugucuuuggacucucaggaaucuuucagaugcugcaacuaaacaggaagggauggaaggucuccuugggacucuuguucagcuucuggguucagaugauauaaauguggucaccugugcagcuggaauucuuucuaaccucacuugcaauaauuauaagaauaagaugauggucugccaagugggugguauagaggcucuugugcguacuguccuucgggcuggugacagggaagacaucacugagccugccaucugugcucuucgucaucugaccagccgacaccaagaagcagagauggcccagaaugcaguucgccuucacuauggacuaccaguugugguuaagcucuuacacccaccaucccacuggccucugauaaaggcuacuguuggauugauucgaaaucuugcccuuuguccagcaaaucaugcaccuuugcgugagcagggugccauuccacgacuaguucaguugcuuguucgugcacaucaggauacccagcgccguacguccaugggugggacacagcagcaauuuguggaggggguccgcauggaagaaauaguugaagguuguacuggagcccuucacauccuagcucgggauguucacaaccgaauuguaaucagaggacuaaauaccauuccauuguuugugcagcugcuuuauucucccauugaaaacauccaaagaguagcugcagggguccucugugaacuugcucaggacaaggaagcugcagaagcgauugaagcugagggagccacagcuccucugacagaguuacuucacucuaggaaugaagguguggcgacguaugcagcugcuguuuuguuccgaaugucugaggacaagccacaagauuacaagaaacggcuuucaguugagcugaccagcucucucuucagaacggagccaauggcuuggaaugagacugcggaucuuggacuugauauuggugcccagggagaaccccuuggauaucgccaggaugauccuagcuaucguucuuuucacucugguggauauggccaggaugccuuggguauggaccccaugauggaacaugagauggguggccaccacccuggugcugacuauccaguugaugggcugccagaucugggacaugcccaggaccucauggaugggcugccuccaggugauagcaaucagcuggccugguuugauacugaccuguaaaucauccuuuagguaagaaguuuuaaaaagccaguuuggguaaaauacuuuuacucugccuacagaauuucagaaagacuugguugguagggugggagugauuuaggcuauuuguaaaucugccacaaaaacagguauauacuuugaaaggagaugucuuggaacaucggaauguucucagauuucugguuguuaugugaucauguguggaaguuauuaacuuuaauguuuuuugccacagcuuuugcaacuuaauacucaaaugaguaacauuugcuguuuuaaacauuaauagcagccuuucucucuuuauacagcuguauugucugaacuugcauugugauuggccuguagaguugcugagagggcucgaggggugggcugguaucucagaaagugccugacacacuaaccaagcugaguuuccuaugggaacaauugaaguaaacuuuuuguucugguccuuuuuggucgaggaguaacaauacaaauggauuuugggagugacucaagaagugaagaaugcacaagaauggaucacaagauggaauuuaucaaacccuagccuugcuuguuaaauuuuuuuuuuuuuuaauaucuguaaugguacugacuuugcuugcuuugaaguagcucuuuaauuuuuuuuuuuuuuuuuuugcaguaacuguuaguuuaagucucucguaguguuaaguuauagugaauacugcuacagcaauuucuaauuuuuaagaauugaguaaugguguagaacacuaauucauaaucacucuaauaauuguaaucugaauaaaguguaacauuguguagccuuuuuguauaaaauagacaaauagaaaaugguccaauuaguuuccuuuuuaauaugcuuaaaauaagcagguggaucuauuucauguuuuugaucaaaaacuuuauuugggauauguauggguaggguaaaucaguaagagguguuauuuggaaccuuguuuuggacaguuuaccaguugccuuuuaucccaaaguuguuguaaccugcugugauacaaugcuucaagagaaaaugcgguuauaaaaaaugguucagaauuaaacuuuuaauucauucgauugugucacucuuuuuuuuucuugucauuaaauggugagggugguaaguuuauccaacucaacuuuauacuga

SEQ ID NO: 102 - DNA, 3671 bp, Macaca mulatta
  cDNA of rhesus monkey CTNNB1
aaagagggacggaauacugcagugcuagggugcuggaguccagaaaugauuuuuuucgaugaauuuaggauuuugccgcaguauacaucugcagucccagaauccuccggguauuugaaguauaccauacaacuguuuugaaaauccagcguggacaauggcuacucaagcugauuugauggaguuggacauggccauggaaccagacagaaaagcggcuguuagucacuggcagcaacagucuuaccuggacucuggaauccauucuggugccacuaccacagcuccuucucugagugguaaaggcaauccugaggaagaggauguggauaccucccaaguccuguaugagugggaacagggauuuucucaguccuucacucaagaacaaguagcugauauugauggacaguaugcaaugacucgagcucagaggguacgagcugcuauguucccugagacauuagaugagggcaugcagaucccaucuacacaguuugaugcugcucaucccacuaauguccagcguuuggcugaaccaucacagaugcugaaacaugcaguuguaaacuugauuaacuaucaagaugaugcagaacuugccacacgugcaaucccugaacugacaaaacugcuaaaugaugaggaccagguggugguuaauaaggcugcaguuaugguccaucagcuuucuaaaaaggaagcuuccagacacgcuaucaugcguucuccucagauggugucugcuauuguacguaccaugcagaauacaaaugauguagaaacagcucguuguaccgcugggaccuugcauaaccuuucccaucaucgggagggcuuguuggccaucuuuaagucuggaggcauuccugcccuggugaaaaugcuugguucaccaguggauucuguguuguuuuaugccauuacaacucuccacaaccuuuuauuacaucaagaaggagcuaaaauggcagugcguuuagcuggcgggcuacagaaaaugguugccuugcucaacaaaacaaacguuaaauucuuggcuauuacgacagacugccuucaaauuuuagcauauggcaaccaagaaagcaagcugaucauacuggcuagugguggaccccaagcuuuaguaaauauaaugaggaccuauacuuaugagaaacuacuguggaccacaagcagagugcugaaggugcuauccgucugcucuaguaauaagccagcuauuguagaagcugguggaaugcaagcuuuaggacuucaccugacagauccaagucaacgucuuguucagaacugucuuuggacucucaggaaucuuucagaugcugcaacuaaacaggaagggauggaaggucuccuugggacucuuguucagcuucuggguucagaugauauaaauguggucaccugugcagcuggaauucuuucuaaccucacuugcaauaauuauaagaauaagaugauggucugccaagugggugguauagaggcucuugugcguacuguccuucgggcuggugacagggaagacaucacugagccugccaucugugcucuucgucaucugaccagccgacaccaagaagcagagauggcccagaaugcaguucgccuucacuauggacuaccaguugugguuaagcucuuacacccaccaucccacuggccucugauaaaggcuacuguuggauugauucgaaaucuugcccuuuguccagcaaaucaugcaccuuugcgugagcagggugccauuccacgacuaguucaguugcuuguucgugcacaucaggauacccagcgccguacguccaugggugggacacagcagcaauuuguggaggggguccgcauggaagaaauaguugaagguuguacuggagcccuucacauccuagcucgggauguucacaaccgaauuguaaucagaggacuaaauaccauuccauuguuugugcagcugcuuuauucucccauugaaaacauccaaagaguagcugcagggguccucugugaacuugcucaggacaaggaagcugcagaagcgauugaagcugagggagccacagcuccucugacagaguuacuucacucuaggaaugaagguguggcgacguaugcagcugcuguuuuguuccgaaugucugaggacaagccacaagauuacaagaaacggcuuucaguugagcugaccagcucucucuucagaacggagccaauggcuuggaaugagacugcggaucuuggacuugauauuggugcccagggagaaccccuuggauaucgccaggaugauccuagcuaucguucuuuucacucugguggauauggccaggaugccuuggguauggaccccaugauggaacaugagauggguggccaccacccuggugcugacuauccaguugaugggcugccagaucugggacaugcccaggaccucauggaugggcugccuccaggugauagcaaucagcuggccugguuugauacugaccuguaaaucauccuuuagguaagaaguuuuaaaaagccaguuuggguaaaauacuuuuacucugccuacagaauuucagaaagacuugguugguagggugggagugauuuaggcuauuuguaaaucugccacaaaaacagguauauacuuugaaaggagaugucuuggaacaucggaauguucucagauuucugguuguuaugugaucauguguggaaguuauuaacuuuaauguuuuuugccacagcuuuugcaacuuaauacucaaaugaguaacauuugcuguuuuaaacauuaauagcagccuuucucucuuuauacagcuguauugucugaacuugcauugugauuggccuguagaguugcugagagggcucgaggggugggcugguaucucagaaagugccugacacacuaaccaagcugaguuuccuaugggaacaauugaaguaaacuuuuuguucugguccuuuuuggucgaggaguaacaauacaaauggauuuugggagugacucaagaagugaagaaugcacaagaauggaucacaagauggaauuuaucaaacccuagccuugcuuguuaaauuuuuuuuuuuuuuaauaucuguaaugguacugacuuugcuugcuuugaaguagcucuuuaauuuuuuuuuuuuuuuuuuugcaguaacuguuaguuuaagucucucguaguguuaaguuauagugaauacugcuacagcaauuucuaauuuuuaagaauugaguaaugguguagaacacuaauucauaaucacucuaauaauuguaaucugaauaaaguguaacauuguguagccuuuuuguauaaaauagacaaauagaaaaugguccaauuaguuuccuuuuuaauaugcuuaaaauaagcagguggaucuauuucauguuuuugaucaaaaacuuuauuugggauauguauggguaggguaaaucaguaagagguguuauuuggaaccuuguuuuggacaguuuaccaguugccuuuuaucccaaaguuguuguaaccugcugugauacaaugcuucaagagaaaaugcgguuauaaaaaaugguucagaauuaaacuuuuaauucauucgauugugucacucuuuuuuuuucuugucauuaaauggugagggugguaaguuuauccaacucaacuuuauacuga

SEQ ID NO: 103 - DNA, 3792 bp, Macaca mulatta
  cDNA of rhesus monkey CTNNB1
cggcggcggcaggauacggcagcuuuugggcgacuuauaagagcuccuugugcggcgccauuuuaagccucuuggucuguggcagccguguuggcccggccccgagagcggagagcgaggggaggcggagacggaggaagguccgaggagcagcuucaguccucgccgagccgccaccgcaggucgaggacggucggacucccgcgacgggaggagccuguuccccugaggguauuugaaguauaccauacaacuguuuugaaaauccagcguggacaauggcuacucaagcugauuugauggaguuggacauggccauggaaccagacagaaaagcggcuguuagucacuggcagcaacagucuuaccuggacucuggaauccauucuggugccacuaccacagcuccuucucugagugguaaaggcaauccugaggaagaggauguggauaccucccaaguccuguaugagugggaacagggauuuucucaguccuucacucaagaacaaguagcugauauugauggacaguaugcaaugacucgagcucagaggguacgagcugcuauguucccugagacauuagaugagggcaugcagaucccaucuacacaguuugaugcugcucaucccacuaauguccagcguuuggcugaaccaucacagaugcugaaacaugcaguuguaaacuugauuaacuaucaagaugaugcagaacuugccacacgugcaaucccugaacugacaaaacugcuaaaugaugaggaccagguggugguuaauaaggcugcaguuaugguccaucagcuuucuaaaaaggaagcuuccagacacgcuaucaugcguucuccucagauggugucugcuauuguacguaccaugcagaauacaaaugauguagaaacagcucguuguaccgcugggaccuugcauaaccuuucccaucaucgggagggcuuguuggccaucuuuaagucuggaggcauuccugcccuggugaaaaugcuugguucaccaguggauucuguguuguuuuaugccauuacaacucuccacaaccuuuuauuacaucaagaaggagcuaaaauggcagugcguuuagcuggcgggcuacagaaaaugguugccuugcucaacaaaacaaacguuaaauucuuggcuauuacgacagacugccuucaaauuuuagcauauggcaaccaagaaagcaagcugaucauacuggcuagugguggaccccaagcuuuaguaaauauaaugaggaccuauacuuaugagaaacuacuguggaccacaagcagagugcugaaggugcuauccgucugcucuaguaauaagccagcuauuguagaagcugguggaaugcaagcuuuaggacuucaccugacagauccaagucaacgucuuguucagaacugucuuuggacucucaggaaucuuucagaugcugcaacuaaacaggaagggauggaaggucuccuugggacucuuguucagcuucuggguucagaugauauaaauguggucaccugugcagcuggaauucuuucuaaccucacuugcaauaauuauaagaauaagaugauggucugccaagugggugguauagaggcucuugugcguacuguccuucgggcuggugacagggaagacaucacugagccugccaucugugcucuucgucaucugaccagccgacaccaagaagcagagauggcccagaaugcaguucgccuucacuauggacuaccaguugugguuaagcucuuacacccaccaucccacuggccucugauaaaggcuacuguuggauugauucgaaaucuugcccuuuguccagcaaaucaugcaccuuugcgugagcagggugccauuccacgacuaguucaguugcuuguucgugcacaucaggauacccagcgccguacguccaugggugggacacagcagcaauuuguggaggggguccgcauggaagaaauaguugaagguuguacuggagcccuucacauccuagcucgggauguucacaaccgaauuguaaucagaggacuaaauaccauuccauuguuugugcagcugcuuuauucucccauugaaaacauccaaagaguagcugcagggguccucugugaacuugcucaggacaaggaagcugcagaagcgauugaagcugagggagccacagcuccucugacagaguuacuucacucuaggaaugaagguguggcgacguaugcagcugcuguuuuguuccgaaugucugaggacaagccacaagauuacaagaaacggcuuucaguugagcugaccagcucucucuucagaacggagccaauggcuuggaaugagacugcggaucuuggacuugauauuggugcccagggagaaccccuuggauaucgccaggaugauccuagcuaucguucuuuucacucugguggauauggccaggaugccuuggguauggaccccaugauggaacaugagauggguggccaccacccuggugcugacuauccaguugaugggcugccagaucugggacaugcccaggaccucauggaugggcugccuccaggugauagcaaucagcuggccugguuugauacugaccuguaaaucauccuuuagguaagaaguuuuaaaaagccaguuuggguaaaauacuuuuacucugccuacagaauuucagaaagacuugguugguagggugggagugauuuaggcuauuuguaaaucugccacaaaaacagguauauacuuugaaaggagaugucuuggaacaucggaauguucucagauuucugguuguuaugugaucauguguggaaguuauuaacuuuaauguuuuuugccacagcuuuugcaacuuaauacucaaaugaguaacauuugcuguuuuaaacauuaauagcagccuuucucucuuuauacagcuguauugucugaacuugcauugugauuggccuguagaguugcugagagggcucgaggggugggcugguaucucagaaagugccugacacacuaaccaagcugaguuuccuaugggaacaauugaaguaaacuuuuuguucugguccuuuuuggucgaggaguaacaauacaaauggauuuugggagugacucaagaagugaagaaugcacaagaauggaucacaagauggaauuuaucaaacccuagccuugcuuguuaaauuuuuuuuuuuuuuaauaucuguaaugguacugacuuugcuugcuuugaaguagcucuuuaauuuuuuuuuuuuuuuuuuugcaguaacuguuaguuuaagucucucguaguguuaaguuauagugaauacugcuacagcaauuucuaauuuuuaagaauugaguaaugguguagaacacuaauucauaaucacucuaauaauuguaaucugaauaaaguguaacauuguguagccuuuuuguauaaaauagacaaauagaaaaugguccaauuaguuuccuuuuuaauaugcuuaaaauaagcagguggaucuauuucauguuuuugaucaaaaacuuuauuugggauauguauggguaggguaaaucaguaagagguguuauuuggaaccuuguuuuggacaguuuaccaguugccuuuuaucccaaaguuguuguaaccugcugugauacaaugcuucaagagaaaaugcgguuauaaaaaaugguucagaauuaaacuuuuaauucauucgauugugucacucuuuuuuuuucuugucauuaaauggugagggugguaaguuuauccaacucaacuuuauacuga

SEQ ID NO: 104 - DNA, 3742 bp, Macaca mulatta
  cDNA of rhesus monkey CTNNB1
cucguaacuguuaccuguaugaauuaagauaaggaguuaugccagaaugguauuugaaguauaccauacaacuguuuugaaaauccagcguggacaauggcuacucaaggcuaccuuuugcuccauuuucugcucacuccuccuaauggcuuggugaaauagcaaacaagccaccagcaggaaucuagucuggaugacugcuucuggagccuggaugcaguaccauucuuccacugauucacugauuugauggaguuggacauggccauggaaccagacagaaaagcggcuguuagucacuggcagcaacagucuuaccuggacucuggaauccauucuggugccacuaccacagcuccuucucugagugguaaaggcaauccugaggaagaggauguggauaccucccaaguccuguaugagugggaacagggauuuucucaguccuucacucaagaacaaguagcugauauugauggacaguaugcaaugacucgagcucagaggguacgagcugcuauguucccugagacauuagaugagggcaugcagaucccaucuacacaguuugaugcugcucaucccacuaauguccagcguuuggcugaaccaucacagaugcugaaacaugcaguuguaaacuugauuaacuaucaagaugaugcagaacuugccacacgugcaaucccugaacugacaaaacugcuaaaugaugaggaccagguggugguuaauaaggcugcaguuaugguccaucagcuuucuaaaaaggaagcuuccagacacgcuaucaugcguucuccucagauggugucugcuauuguacguaccaugcagaauacaaaugauguagaaacagcucguuguaccgcugggaccuugcauaaccuuucccaucaucgggagggcuuguuggccaucuuuaagucuggaggcauuccugcccuggugaaaaugcuugguucaccaguggauucuguguuguuuuaugccauuacaacucuccacaaccuuuuauuacaucaagaaggagcuaaaauggcagugcguuuagcuggcgggcuacagaaaaugguugccuugcucaacaaaacaaacguuaaauucuuggcuauuacgacagacugccuucaaauuuuagcauauggcaaccaagaaagcaagcugaucauacuggcuagugguggaccccaagcuuuaguaaauauaaugaggaccuauacuuaugagaaacuacuguggaccacaagcagagugcugaaggugcuauccgucugcucuaguaauaagccagcuauuguagaagcugguggaaugcaagcuuuaggacuucaccugacagauccaagucaacgucuuguucagaacugucuuuggacucucaggaaucuuucagaugcugcaacuaaacaggaagggauggaaggucuccuugggacucuuguucagcuucuggguucagaugauauaaauguggucaccugugcagcuggaauucuuucuaaccucacuugcaauaauuauaagaauaagaugauggucugccaagugggugguauagaggcucuugugcguacuguccuucgggcuggugacagggaagacaucacugagccugccaucugugcucuucgucaucugaccagccgacaccaagaagcagagauggcccagaaugcaguucgccuucacuauggacuaccaguugugguuaagcucuuacacccaccaucccacuggccucugauaaaggcuacuguuggauugauucgaaaucuugcccuuuguccagcaaaucaugcaccuuugcgugagcagggugccauuccacgacuaguucaguugcuuguucgugcacaucaggauacccagcgccguacguccaugggugggacacagcagcaauuuguggaggggguccgcauggaagaaauaguugaagguuguacuggagcccuucacauccuagcucgggauguucacaaccgaauuguaaucagaggacuaaauaccauuccauuguuugugcagcugcuuuauucucccauugaaaacauccaaagaguagcugcagggguccucugugaacuugcucaggacaaggaagcugcagaagcgauugaagcugagggagccacagcuccucugacagaguuacuucacucuaggaaugaagguguggcgacguaugcagcugcuguuuuguuccgaaugucugaggacaagccacaagauuacaagaaacggcuuucaguugagcugaccagcucucucuucagaacggagccaauggcuuggaaugagacugcggaucuuggacuugauauuggugcccagggagaaccccuuggauaucgccaggaugauccuagcuaucguucuuuucacucugguggauauggccaggaugccuuggguauggaccccaugauggaacaugagauggguggccaccacccuggugcugacuauccaguugaugggcugccagaucugggacaugcccaggaccucauggaugggcugccuccaggugauagcaaucagcuggccugguuugauacugaccuguaaaucauccuuuagguaagaaguuuuaaaaagccaguuuggguaaaauacuuuuacucugccuacagaauuucagaaagacuugguugguagggugggagugauuuaggcuauuuguaaaucugccacaaaaacagguauauacuuugaaaggagaugucuuggaacaucggaauguucucagauuucugguuguuaugugaucauguguggaaguuauuaacuuuaauguuuuuugccacagcuuuugcaacuuaauacucaaaugaguaacauuugcuguuuuaaacauuaauagcagccuuucucucuuuauacagcuguauugucugaacuugcauugugauuggccuguagaguugcugagagggcucgaggggugggcugguaucucagaaagugccugacacacuaaccaagcugaguuuccuaugggaacaauugaaguaaacuuuuuguucugguccuuuuuggucgaggaguaacaauacaaauggauuuugggagugacucaagaagugaagaaugcacaagaauggaucacaagauggaauuuaucaaacccuagccuugcuuguuaaauuuuuuuuuuuuuuaauaucuguaaugguacugacuuugcuugcuuugaaguagcucuuuaauuuuuuuuuuuuuuuuuuugcaguaacuguuaguuuaagucucucguaguguuaaguuauagugaauacugcuacagcaauuucuaauuuuuaagaauugaguaaugguguagaacacuaauucauaaucacucuaauaauuguaaucugaauaaaguguaacauuguguagccuuuuuguauaaaauagacaaauagaaaaugguccaauuaguuuccuuuuuaauaugcuuaaaauaagcagguggaucuauuucauguuuuugaucaaaaacuuuauuugggauauguauggguaggguaaaucaguaagagguguuauuuggaaccuuguuuuggacaguuuaccaguugccuuuuaucccaaaguuguuguaaccugcugugauacaaugcuucaagagaaaaugcgguuauaaaaaaugguucagaauuaaacuuuuaauucauucgauugugucacucuuuuuuuuucuugucauuaaauggugagggugguaaguuuauccaacucaacuuuauacuga

SEQ ID NO: 105 - DNA, 3640 bp, Mus musculus
  cDNA of mouse CTNNB1
gcgcggcggaacgcuccgcgcggagcggcagcggcaggauacacggugccgcgccgcuuauaaaucgcuccuugugcggcgccaucuuaagcccucgcucgguggcggccgcgucagcucguguccugugaagcccgcggcccggggaggcggagacggagcacggugggcgccgagccgucagugcaggaggccgaggccgagcgggcggccgcgagugagcagcgcgcgggccugaggguaccugaagcucagcgcacagcugcugugacaccgcugcguggacaauggcuacucaagcugaccugauggaguuggacauggccauggagccggacagaaaagcugcugucagccacuggcagcagcagucuuacuuggauucuggaauccauucuggugccaccaccacagcuccuucccugaguggcaagggcaacccugaggaagaagauguugacaccucccaaguccuuuaugaaugggagcaaggcuuuucccaguccuucacgcaagagcaaguagcugauauugacgggcaguaugcaaugacuagggcucagaggguccgagcugccauguucccugagacgcuagaugagggcaugcagaucccauccacgcaguuugacgcugcucaucccacuaauguccagcgcuuggcugaaccaucacagauguugaaacaugcaguugucaauuugauuaacuaucaggaugacgcggaacuugccacacgugcaauuccugagcugacaaaacugcuaaacgaugaggaccaggugguaguuaauaaagcugcuguuaugguccaucagcuuuccaaaaaggaagcuuccagacaugccaucaugcgcuccccucagauggugucugccauuguacgcaccaugcagaauacaaaugauguagagacagcucguuguacugcugggacucugcacaaccuuucucaccaccgcgagggcuugcuggccaucuuuaagucugguggcaucccagcgcuggugaaaaugcuugggucaccaguggauucuguacuguucuacgccaucacgacacugcauaaucuccugcuccaucaggaaggagcuaaaauggcagugcgccuagcugguggacugcagaaaaugguugcuuugcucaacaaaacaaacgugaaauucuuggcuauuacaacagacugccuucagaucuuagcuuauggcaaucaagagagcaagcucaucauucuggccagugguggaccccaagccuuaguaaacauaaugaggaccuacacuuaugagaagcuucuguggaccacaagcagagugcugaaggugcugucugucugcucuagcaacaagccggccauuguagaagcuggugggaugcaggcacuggggcuucaucugacagacccaagucagcgacuuguucaaaacugucuuuggacucucagaaaccuuucagaugcagcgacuaagcaggaagggauggaaggccuccuugggacucuagugcagcuucuggguuccgaugauauaaauguggucaccugugcagcuggaauucucucuaaccucacuugcaauaauuacaaaaacaagaugauggugugccaaguggguggcauagaggcucuuguacgcaccguccuucgugcuggugacagggaagacaucacugagccugccaucugugcucuucgucaucugaccagccggcaucaggaagccgagauggcccagaaugccguucgccuucauuauggacugccuguugugguuaaacuccugcacccaccaucccacuggccucugauaaaggcaacuguuggauugauucgaaaccuugcccuuugcccagcaaaucaugcgccuuugcgggaacagggugcuauuccacgacuaguucagcugcuuguacgagcacaucaggacacccaacggcgcaccuccauggguggaacgcagcagcaguuuguggagggcgugcgcauggaggagauaguagaaggguguacuggagcucuccacauccuugcucgggacguucacaaccggauuguaauccgaggacucaauaccauuccauuguuugugcaguugcuuuauucucccauugaaaauauccaaagaguagcugcagggguccucugugaacuugcucaggacaaggaggcugcagaggccauugaagcugagggagccacagcuccccugacagaguuacuccacuccaggaaugaaggcguggcaacauacgcagcugcuguccuauuccgaaugucugaggacaagccacaggauuacaagaagcggcuuucagucgagcugaccaguucccucuucaggacagagccaauggcuuggaaugagacugcagaucuuggacuggacauuggugcccagggagaagcccuuggauaucgccaggaugaucccagcuaccguucuuuucacucugguggauacggccaggaugccuuggggauggacccuaugauggagcaugagauggguggccaccacccuggugcugacuauccaguugaugggcugccugaucugggacacgcccaggaccucauggaugggcugcccccaggugauagcaaucagcuggccugguuugauacugaccuguaaaucguccuuuagguaagaaagcuuauaaaagccagugugggugaauacuuuacucugccugcagaacuccagaaagacuugguagggugggaaugguuuuaggccuguuuguaaaucugccaccaaacagauacauaccuuggaaggagauguucauguguggaaguuucucacguugauguuuuugccacagcuuuugcagcguuauacucagaugaguaacauuugcuguuuucaacauuaauagcagccuuucucucuauacagcuguagugucugaacgugcauugugauuggccuguagaguugcugagagggcucgaggggugggcugguaucucagaaagugccugacacacuaaccaagcugaguuuccuaugggaacagucgaaguacgcuuuuuguucugguccuuuuuggucgaggaguaacaauacaaauggauuuggggagugacucacgcagugaagaaugcacacgaauggaucacaagauggcguuaucaaacccuagccuugcuuguucuuuguuuuaauaucuguaguggugcugacuuugcuugcuuuuauuuuuugcaguaacuguuaguuuuuaaguaguguuauguucuagugaaccugcuacagcaauuucugauuucuaagaaccgaguaaugguguagaacacuaauucauaaucacgcuaauuguaaucuggagacguguaacauuguguagccuuuuguauaaauagacagauagaaaugguccgauuaguuuccuuuuuaauaugcuuaaaauaagcagguggaucuauuucauguuuuugaacaaaaacuuuaucggggauacgugcgguaggguaaaucaguaagagguguuauuugagccuuguuuuggacaguauaccaguugccuuuuaucccaaaguuguuguaaccugcugugauacaaugcuucaacagaugcgguuauagaaaugguucagaauuaaacuuuuaauucauucaaaaaaaaaaaaaaaaaa

SEQ ID NO: 106 - DNA, 3440 bp, Mus musculus
  cDNA of mouse CTNNB1
cgucagugcaggaggccgaggccgagcgggcggccgcgagguaccugaagcucagcgcacagcugcugugacaccgcugcguggacaauggcuacucaagcugaccugauggaguuggacauggccauggagccggacagaaaagcugcugucagccacuggcagcagcagucuuacuuggauucuggaauccauucuggugccaccaccacagcuccuucccugaguggcaagggcaacccugaggaagaagauguugacaccucccaaguccuuuaugaaugggagcaaggcuuuucccaguccuucacgcaagagcaaguagcugauauugacgggcaguaugcaaugacuagggcucagaggguccgagcugccauguucccugagacgcuagaugagggcaugcagaucccauccacgcaguuugacgcugcucaucccacuaauguccagcgcuuggcugaaccaucacagauguugaaacaugcaguugucaauuugauuaacuaucaggaugacgcggaacuugccacacgugcaauuccugagcugacaaaacugcuaaacgaugaggaccaggugguaguuaauaaagcugcuguuaugguccaucagcuuuccaaaaaggaagcuuccagacaugccaucaugcgcuccccucagauggugucugccauuguacgcaccaugcagaauacaaaugauguagagacagcucguuguacugcugggacucugcacaaccuuucucaccaccgcgagggcuugcuggccaucuuuaagucugguggcaucccagcgcuggugaaaaugcuugggucaccaguggauucuguacuguucuacgccaucacgacacugcauaaucuccugcuccaucaggaaggagcuaaaauggcagugcgccuagcugguggacugcagaaaaugguugcuuugcucaacaaaacaaacgugaaauucuuggcuauuacaacagacugccuucagaucuuagcuuauggcaaucaagagagcaagcucaucauucuggccagugguggaccccaagccuuaguaaacauaaugaggaccuacacuuaugagaagcuucuguggaccacaagcagagugcugaaggugcugucugucugcucuagcaacaagccggccauuguagaagcuggugggaugcaggcacuggggcuucaucugacagacccaagucagcgacuuguucaaaacugucuuuggacucucagaaaccuuucagaugcagcgacuaagcaggaagggauggaaggccuccuugggacucuagugcagcuucuggguuccgaugauauaaauguggucaccugugcagcuggaauucucucuaaccucacuugcaauaauuacaaaaacaagaugauggugugccaaguggguggcauagaggcucuuguacgcaccguccuucgugcuggugacagggaagacaucacugagccugccaucugugcucuucgucaucugaccagccggcaucaggaagccgagauggcccagaaugccguucgccuucauuauggacugccuguugugguuaaacuccugcacccaccaucccacuggccucugauaaaggcaacuguuggauugauucgaaaccuugcccuuugcccagcaaaucaugcgccuuugcgggaacagggugcuauuccacgacuaguucagcugcuuguacgagcacaucaggacacccaacggcgcaccuccauggguggaacgcagcagcaguuuguggagggcgugcgcauggaggagauaguagaaggguguacuggagcucuccacauccuugcucgggacguucacaaccggauuguaauccgaggacucaauaccauuccauuguuugugcaguugcuuuauucucccauugaaaauauccaaagaguagcugcagggguccucugugaacuugcucaggacaaggaggcugcagaggccauugaagcugagggagccacagcuccccugacagaguuacuccacuccaggaaugaaggcguggcaacauacgcagcugcuguccuauuccgaaugucugaggacaagccacaggauuacaagaagcggcuuucagucgagcugaccaguucccucuucaggacagagccaauggcuuggaaugagacugcagaucuuggacuggacauuggugcccagggagaagcccuuggauaucgccaggaugaucccagcuaccguucuuuucacucugguggauacggccaggaugccuuggggauggacccuaugauggagcaugagauggguggccaccacccuggugcugacuauccaguugaugggcugccugaucugggacacgcccaggaccucauggaugggcugcccccaggugauagcaaucagcuggccugguuugauacugaccuguaaaucguccuuuagguaagaaagcuuauaaaagccagugugggugaauacuuuacucugccugcagaacuccagaaagacuugguagggugggaaugguuuuaggccuguuuguaaaucugccaccaaacagauacauaccuuggaaggagauguucauguguggaaguuucucacguugauguuuuugccacagcuuuugcagcguuauacucagaugaguaacauuugcuguuuucaacauuaauagcagccuuucucucuauacagcuguagugucugaacgugcauugugauuggccuguagaguugcugagagggcucgaggggugggcugguaucucagaaagugccugacacacuaaccaagcugaguuuccuaugggaacagucgaaguacgcuuuuuguucugguccuuuuuggucgaggaguaacaauacaaauggauuuggggagugacucacgcagugaagaaugcacacgaauggaucacaagauggcguuaucaaacccuagccuugcuuguucuuuguuuuaauaucuguaguggugcugacuuugcuugcuuuuauuuuuugcaguaacuguuaguuuuuaaguaguguuauguucuagugaaccugcuacagcaauuucugauuucuaagaaccgaguaaugguguagaacacuaauucauaaucacgcuaauuguaaucuggagacguguaacauuguguagccuuuuguauaaauagacagauagaaaugguccgauuaguuuccuuuuuaauaugcuuaaaauaagcagguggaucuauuucauguuuuugaacaaaaacuuuaucggggauacgugcgguaggguaaaucaguaagagguguuauuugagccuuguuuuggacaguauaccaguugccuuuuaucccaaaguuguuguaaccugcugugauacaaugcuucaacagaugcgguuauagaaaugguucagaauuaaacuuuuaauucauucaaaaaaaaaaaaaaaaaa

(19) United States
(12) Patent Application Publication
(10) Pub. No.: US 2015/0291956 A1
(43) Pub. Date: Oct. 15, 2015

(54) RNA TARGETED TO BETA CATENIN

(71) Applicant: PHASERX, INC., Seattle, WA (US)

(72) Inventors: Markus Hossbach, Kulmbach (DE); Jochen Deckert, Kulmbach (DE)

(73) Assignee: PHASERX, INC., Seattle, WA (US)

(21) Appl. No.: 14/437,923

(22) PCT Filed: Feb. 19, 2013
(86) PCT No.: PCT/US13/26624

§ 371 Date: Apr. 23, 2015

Related U.S. Application Data
(60) Provisional application No. 61/718,313, filed on Oct. 25, 2012.

(51) Int. Cl. C12N 15/113

(52) U.S. Cl. CPC C12N 15/113; C12N 2310/11; C12N 2310/111; C12N 2310/322; C12N 2310/3533; C12N 2310/315

(57) ABSTRACT

A double stranded RNA (dsRNA) molecule targeted to beta catenin includes a duplex region having a sense region and an antisense region at least substantially complementary to the sense region. The sense region and the antisense region each have between 18 and 30 nucleotides. The antisense region includes a nucleotide sequence that is fully complementary to at least 15 contiguous nucleotides of any one of SEQ ID NOs: 1-24.

### RELATED APPLICATION

This application claims the benefit of U.S. provisional application No. 61/718,313 filed on Oct. 25, 2012, which provisional patent application is hereby incorporated herein in its entirety to the extent that it does not conflict with the present disclosure.

### FIELD

The present disclosure relates to compounds and compositions that target Beta catenin expression and methods of use thereof. More particularly, this disclosure relates to RNA compounds capable of selective hybridization with nucleic acids encoding human Beta catenin, and which are capable of modulating expression of Beta catenin.

### SEQUENCE LISTING

This application contains a Sequence Listing electronically submitted via EFS-Web to the United States Patent and Trademark Office as text filed named “120806_CTNNB1_siRNAs_IP_sequence_lising.txt” and having a size of 85 kb. The information contained in the Sequence Listing is hereby incorporated herein by reference.

### BACKGROUND

Beta catenin has been implicated in a number of cancers, including basal cell carcinoma, colorectal cancer, pilomatrixoma, medullablastoma, and ovarian cancer, as well as adenomatous polyposis of the colon. The gene encoding beta catenin may act as an oncogene in some cases. For example, an increase in beta catenin has been observed in people with basal cell carcinoma and can increase proliferation in related tumors. In addition, mutations in the gene encoding beta catenin have been observed in various cancers.

Potential therapeutic agents for inhibiting the synthesis of Beta catenin have been proposed including antibodies, antisense oligonucleotides, small inhibitory RNAs (siRNAs), and chemical inhibitors. By way of example, U.S. Pat. No. 6,066,500, entitled ANTISENSE MODULATION OF BETA CATENIN EXPRESSION, issued May 23, 2000 and naming C. Frank Bennett and Lex M. Cowsert as inventors, discloses, among other things, antisense compounds that target human beta catenin and inhibit beta catenin mRNA levels. By way of further example, U.S. Pat. No. 8,198,427, entitled SIRNA TARGETING CATENIN, BETA-1 (CTNNB1), issued Jun. 12, 2012 and naming A. Khvorova et al. as inventors, discloses, among other things, siRNAs that target nucleotide sequences of beta catenin. Of these potential therapeutic agents, polynucleotides, such as antisense compounds and siRNAs, are of interest because of their specificity.

When introduced to cells, certain short sequence-specific RNA duplexes, such as those about 17-30 base pairs in length, can result in cleavage of target mRNA. The interference effect of such small inhibitory RNAs (siRNAs) may be long lasting and may be detectable after many rounds of cell divisions. Accordingly, siRNA may serve as an effective tool for inhibiting expression of specific genes and may be valuable as therapeutic agents against diseases that are caused by over-expression or misexpression of genes or diseases brought about by genes that contain mutations.

Different siRNAs targeting the same gene product may vary with regard to specificity, efficacy, stability, and the like. Accordingly, it is desirable to continue developing new siRNAs as the properties of the new siRNAs may have one or more advantages relative to previously developed siRNA that target the same gene product.

### BRIEF SUMMARY

The present disclosure describes, among other things, double stranded RNAs, such as siRNAs, that target Beta catenin. The dsRNAs described herein may be used to silence expression of Beta catenin. In embodiments, a given dsRNA may be used to silence Beta catenin in cells from a variety of species, such as mouse, monkey and human, so a dsRNA tested in preclinical studies in, e.g. mice or monkeys, can be used in clinical studies or for therapeutic purposes in humans. In embodiments, the dsRNAs are directed to regions of Beta catenin mRNA believed to be free from, or at least having a low likelihood of, single nucleotide polymorphisms so that the dsRNAs are effective across many individuals in a population.

In embodiments described herein, a double stranded RNA molecule targeted to Beta catenin includes a duplex region having a sense region and an antisense region at least substantially complementary to the sense region. The sense region and the antisense region each have between 18 and 30 nucleotides. The antisense region includes a nucleotide sequence that is fully complementary to at least 15 contiguous nucleotides of any one of SEQ ID NOs:1-24. Preferably, the dsRNA inhibits expression of Beta catenin by about 90% or more. In embodiments, the dsRNA may be used to treat cancer in a patient, such as a patient having liver cancer.

Advantages of one or more of the various embodiments presented herein over prior cell culture compositions and methods will be readily apparent to those of skill in the art based on the following detailed description and examples.

### DETAILED DESCRIPTION

In the following detailed description and examples several specific embodiments are provided to illustrate the compounds, compositions, and methods described herein. It is to be understood that other embodiments are contemplated and may be made without departing from the scope or spirit of the present disclosure. The following detailed description and examples, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used herein, the singular forms “a”, “an”, and “the” encompass embodiments having plural referents, unless the content clearly dictates otherwise. In addition, plural referents may refer to singular forms herein, unless content clearly dictates otherwise.

As used herein, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising,” and the like.

“G,” “C,” “A”, “U” and “T” or “dT” respectively, each generally stand for a nucleotide that contains guanine, cytosine, adenine, uracil and deoxythymidine as a base, respectively. However, the term “ribonucleotide” or “nucleotide” can also refer to a modified nucleotide, as further described herein below, or a surrogate replacement moiety. Sequences comprising such replacement moieties are embodiments of the dsRNAs described herein.

As used herein, the terms “strand comprising a sequence,” “region comprising a sequence,” or the like refers to an oligonucleotide comprising a chain of nucleotides that is described by the sequence referred to using the standard nucleotide nomenclature. However, as detailed herein, such a “strand comprising a sequence,” “region comprising a sequence,” or the like may comprise modifications, like modified nucleotides.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence. “Complementary” sequences, as used herein, may also include, or be formed entirely from, non-Watson-Crick base pairs or base pairs formed from non-natural and modified nucleotides, in as far as the above requirements with respect to their ability to hybridize are fulfilled.

Sequences referred to as “fully complementary” comprise base-pairing of the oligonucleotide or polynucleotide comprising the first nucleotide sequence to the oligonucleotide or polynucleotide comprising the second nucleotide sequence over the entire length of the first and second nucleotide sequence.

The terms “complementary”, “fully complementary” and “substantially complementary” herein may be used with respect to the base matching between the sense strand or region and the antisense strand or region of a dsRNA, or between the antisense strand or region of a dsRNA and a target sequence, as will be understood from the context of their use. Where a first sequence is referred to as “substantially complementary” with respect to a second sequence herein, the two sequences can be fully complementary, or they may form one or more, but preferably not more than 6 mismatched base pairs upon hybridization. “Substantially complementary” preferably means at least 85% (e.g., at least about 90% or at least about 95%) of the overlapping nucleotides in sense and antisense strands or regions are complementary.

The term “double-stranded RNA”, “dsRNA molecule”, or “dsRNA”, as used herein, refers to a ribonucleic acid molecule, or complex of ribonucleic acid molecules, having a duplex structure (also referred to herein as “duplex region”) comprising two anti-parallel and substantially complementary or complementary nucleic acid strands or regions. The term “antisense strand” refers to the strand of a dsRNA which includes a region (an “antisense region”) that is substantially complementary or complementary to a target sequence. As used herein, the term “region of complementarity” refers to the region on the antisense strand that is substantially complementary or fully complementary to a sequence, for example a target sequence. Where the region of complementarity is not fully complementary to the target sequence, the mismatches are most tolerated outside nucleotides 2-7 of the 5′ terminus of the antisense strand.

The term “sense strand,” as used herein, refers to the strand of a dsRNA that includes a region (a “sense region”) that is substantially complementary or fully complementary to a region of the antisense strand.

As used herein, “target sequence” refers to a contiguous portion of the nucleotide sequence of an mRNA molecule formed during the transcription of a CTNNB1 gene, including mRNA that is a product of RNA processing of a primary transcription product.

As used herein, “CTNNB1” refers to catenin, beta-1. CTNNB1 may be human (Homo sapiens), rhesus monkey (Macaca mulatta) CTNNB1, mouse (Mus musculus) CTNNB1, rat (Rattus novegicus) CTNNB1, or the like. Preferably, a CTNNB1 target sequence is a sequence present in a human CTNNB1 sequence.

The terms “silence”, “inhibit the expression of” and “knock down”, in as far as they refer to a CTNNB1 gene, herein refer to the at least partial suppression of the expression of a CTNNB1 gene.

The term “off target” as used herein refers to all non-target mRNAs of the transcriptome that are predicted by in silico methods to hybridize to the described dsRNAs based on sequence complementarity. The dsRNAs described herein preferably do specifically inhibit the expression of any gene other than the CTNNB1 gene, i.e. do not inhibit the expression of any off-target genes.

The present disclosure describes, among other things, dsRNA directed to a CTNNB1 sequence. Preferably, the dsRNA is directed a nucleotide sequence of an mRNA molecule formed during the transcription of a CTNNB1 gene, such as mRNA that is a product of RNA processing of a primary transcription product. Preferably, the dsRNA is capable of silencing, inhibiting the expression of, or knocking down CTNNB1 in a cell when the dsRNA is introduced into the cell.

The ability of a dsRNA to inhibit expression of CTNNB1 may be determined in an in vitro assay. The term “in vitro” as used herein includes but is not limited to cell culture assays. A person skilled in the art can readily determine such an inhibition rate and related effects. By way of example, the efficacy of a dsRNA to inhibit expression of CTNNB1 may be evaluated by comparing the amount of mRNA transcribed from a CTNNB1 gene isolated from a first cell or group of cells in which a CTNNB1 gene is transcribed and which has or have been treated such that the expression of a CTNNB1 gene is inhibited to a second cell or group of cells substantially identical to the first cell or group of cells but which has or have not been so treated (control cells). The degree of inhibition is may be expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the CTNNB1 gene transcription, e.g. the amount of protein encoded by a CTNNB1 gene which is secreted by a cell, the number of cells displaying a certain phenotype, or the decrease in a downstream gene, as measured by mRNA or protein levels, within the wnt signalling cascade such as glutamine synthetase.

In embodiments, the dsRNA inhibits expression of CTNNB1 by about 20% or more, such as by about 60% or more, 70% or more, 80% or more, or 90% or more.

In embodiments, the dsRNA comprises an antisense region that is fully complementary to at least 15 contiguous nucleotides of any one of SEQ ID NOs:1-24, which are discussed in more detail in the Examples that follow. SEQ ID NOs:1-24 are selected as nucleotide sequences that are present in human, monkey, and mouse CTNNB1 mRNA while SEQ ID NOs: 1-4, 6, 7, 10, 13, 15, 16, 18-20 and 22-24 are additionally present in rat. Accordingly, a given dsRNA may be useful both for preclinical analysis in mice, rats or monkeys and for clinical studies or therapeutic use in humans. SEQ ID NOs:1-24 are also selected as sequences believed to have low likelihood or to be free from single nucleotide polymorphisms, which should allow the dsRNAs to be effective in many individuals within a population.

The dsRNAs described herein include a duplex region having a sense region and an anti-parallel and substantially complementary or complementary antisense region. The sense region and the antisense region each may include any suitable number or nucleotides. In embodiments, the sense region and the antisense region each contain between 18 and 30 nucleotides, such as between 18 and 25 nucleotides, between 18 and 20 nucleotides, or about 19 nucleotides.

The antisense region preferably comprises or consists of a nucleotide sequence that is fully complementary to at least 15 contiguous nucleotides of any one of SEQ ID NOs:1-24 (preferably SEQ ID NOs:1-9, 11-15, 17-19, and 21; more preferably SEQ ID NOs:1-8, and even more preferably SEQ ID NOs:1-6), such as at least 16 contiguous nucleotides of any one of SEQ ID NOs:1-24 (preferably SEQ ID NOs:1-9, 11-15, 17-19, and 21; more preferably SEQ ID NOs:1-8, and even more preferably SEQ ID NOs:1-6), at least 17 contiguous nucleotides of any one of SEQ ID NOs:1-24 (preferably SEQ ID NOs:1-9, 11-15, 17-19, and 21; more preferably SEQ ID NOs:1-8, and even more preferably SEQ ID NOs:1-6), or at least 18 contiguous nucleotides of any one of SEQ ID NOs:1-24 (preferably SEQ ID NOs:1-9, 11-15, 17-19, and 21; more preferably SEQ ID NOs:1-8, and even more preferably SEQ ID NOs:1-6). In embodiments, the antisense region comprises or consists of a nucleotide sequence that is fully complementary to any one of SEQ ID NOs:1-24 (preferably SEQ ID NOs:1-9, 11-15, 17-19, and 21; more preferably SEQ ID NOs:1-8, and even more preferably SEQ ID NOs: 1-6).

In embodiments, the antisense region comprises at least 15 consecutive nucleotides of any one of SEQ ID NOs:73-96 (preferably SEQ ID NOs:73-81, 83-87, 89-91, and 93; more preferably SEQ ID NOs:73-80, and even more preferably SEQ ID NOs:73-78), which are discussed in more detail below in the Examples. For example, the antisense region may comprise at least 16 consecutive nucleotides of any one of SEQ ID NOs:73-96 (preferably SEQ ID NOs:73-81, 83-87, 89-91, and 93; more preferably SEQ ID NOs:73-80, and even more preferably SEQ ID NOs:73-78), at least 17 consecutive nucleotides of any one of SEQ ID NOs:73-96 (preferably SEQ ID NOs:73-81, 83-87, 89-91, and 93; more preferably SEQ ID NOs:73-80, and even more preferably SEQ ID NOs:73-78), or at least 18 consecutive nucleotides of any one of SEQ ID NOs:73-96 (preferably SEQ ID NOs:73-81, 83-87, 89-91, and 93; more preferably SEQ ID NOs:73-80, and even more preferably SEQ ID NOs:73-78). In embodiments, the antisense region consists of a nucleotide sequence according to any one of SEQ ID NOs:73-96 (preferably SEQ ID NOs:73-81, 83-87, 89-91, and 93; more preferably SEQ ID NOs:73-80, and even more preferably SEQ ID NOs:73-78).

The sense region and the antisense region may be present on a single strand or on separate strands (the sense strand and the antisense strand, respectively). Where the two regions are part of one larger molecule, and therefore are connected by an uninterrupted chain of nucleotides between the 3′-end of one region and the 5′-end of the respective other region forming the duplex region, the two regions may be connected by a hairpin loop. Where the two regions are connected covalently by means other than an uninterrupted chain of nucleotides between the 3′-end of one region and the 5′-end of the respective other region forming the duplex region, the connecting structure may be a linker. The RNA strands or regions may have the same or a different number of nucleotides.

In addition to the duplex structure, a dsRNA may optionally comprise one or more nucleotide overhangs. A “nucleotide overhang” refers to the unpaired nucleotide or nucleotides that protrude from the duplex structure of a dsRNA when a 3′-end of one strand or region of the dsRNA extends beyond the 5′-end of the other strand or region, or vice versa. Accordingly, the overhanging nucleotides are typically not directly involved in the RNA double helical structure normally formed by the herein defined pair of sense strand or region and antisense strand or region” Preferably, the overhangs are located in the 3′-end of the strand or region.

The nucleotides in said “overhangs” may comprise between 0 and 5 nucleotides, whereby “0” means no additional nucleotide(s) that form(s) an “overhang” and whereas “5” means five additional nucleotides on the individual strands of the dsRNA duplex. In embodiments, an overhang consists of one or two nucleotides. One or more of the nucleotides in the overhang may be fully complementary to the mRNA of the target gene. In embodiments, all of the nucleotides in the overhang are fully complementary to the mRNA of the target gene.

An overhang may contain any suitable nucleotide. In embodiments, the overhang consists of two uracil nucleotides. In embodiments, the overhang consists of two dT (deoxythymidine) nucleotides. The nucleotides may be modified in any suitable manner.

In embodiments, a dsRNA has only one overhang. In some embodiments where the sense region and the antisense region are present on a single strand, the dsRNA has only one strand. In embodiments, a dsRNA has two overhangs. In embodiments, a dsRNA contains no overhangs or is blunt ended. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

As illustrated by SEQ ID NOs:49-96, which are described in more detail below in the Examples that follow, one or more or all of the nucleotides in a strand, region, or dsRNA as described herein may be modified. A nucleotide may be modified in any suitable manner, such as those well-known in the art. For example, nucleotides may have modifications in the chemical structure of the base, sugar or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety such as methyl. Modified nucleotides are also meant to include nucleotides with bases such as inosine, queuosine, xanthine, sugars such as 2′-methyl ribose, non-natural phosphodiester linkages such as methylphosphonates, phosphorothioates and peptides.

Nucleotides or oligonucleotides as described herein may be modified by chemically linking to the nucleotide or oligonucleotide to one or more moieties or conjugates which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. It is not necessary for all positions in a given ds RNA to be uniformly modified, and in fact more than one of the aforementioned modifications may be incorporated in a single compound or even at a single nucleoside within an oligonucleotide.

As indicated above, the modifications, in some embodiments, may extend the half-life of a resulting dsRNA. The term “half-life” as used herein is a measure of stability of a compound or molecule and can be assessed by methods known to a person skilled in the art, especially in light of the assays discussed in more detail below in the Examples.

The dsRNA molecules described herein may be conveniently and routinely made through the well-known technique of solid phase synthesis. Any other means for such synthesis known in the art may additionally or alternatively be employed. It is well known to use similar techniques to prepare oligonucleotides such as the phosphorothioates and alkylated derivatives.

In embodiments, the dsRNA molecules described herein are synthesized in vitro and do not include antisense compositions of biological origin, or are genetic vector constructs designed to direct the in vivo synthesis of antisense molecules.

The dsRNAs described herein may be admixed, encapsulated, conjugated or otherwise associated with other molecules, molecule structures or mixtures of compounds, as for example, liposomes and lipids such as those disclosed in U.S. Pat. Nos. 6,815,432, 6,586,410, 6,858,225, 7,811,602, 7,244,448 and 8,158,601, for example, polymeric materials such as those disclosed in U.S. Pat. Nos. 6,835,393, 7,374,778, 7,737,108, 7,718,193, 8,137,695 and United States Patent Applications Publication Nos. 2011/0143434, 2011/0129921, 2011/0123636, 2011/0143435, 2011/0142951, 2012/0021514, 2011/0281934, 2011/0286957 and 2008/0152661, for example, receptor targeted molecules, oral, rectal, topical or other formulations, for assisting in uptake, distribution or absorption.

The dsRNAs described herein encompass any pharmaceutically acceptable salts, esters, or salts of such esters, or any other compound which, upon administration to an animal including a human, is capable of providing (directly or indirectly) the biologically active metabolite or residue thereof.

As used herein, “pharmaceutically acceptable salts” refers to physiologically and pharmaceutically acceptable salts of the dsRNAs described herein: i.e., salts that retain the desired biological activity of the dsRNA and do not impart undesired toxicological effects thereto.

Pharmaceutically acceptable base addition salts may be formed with metals or amines, such as alkali and alkaline earth metals or organic amines Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine. The base addition salts of said acidic compounds may be prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure. As used herein, a “pharmaceutical addition salt” includes a pharmaceutically acceptable salt of an acid form of one of the dsRNAs described herein. These include organic or inorganic acid salts of the amines Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides such as dsRNAs, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The dsRNAs described herein may be used for any suitable purpose, such as for diagnostics, therapeutics, prophylaxis, as research reagents or in kits. For therapeutics, an animal, preferably a human, suspected of having a disease or disorder which can be treated by modulating the expression of CTNNB1 is treated by introducing a dsRNA into a cell of the animal to be treated. Because CTNNB1 has been implicated in a variety of cancers, a dsRNA as described herein may be used to treat a patient having a cancer that may benefit from inhibition of CTNNB1 expression. Such cancers may include basal cell carcinoma, colorectal cancer, pilomatrixoma, medullablastoma, liver cancer and ovarian cancer. Examples of liver cancers that may be treated via a dsRNA as described herein include hepatocellular carcinoma and cholangiocarcinoma. Additionally, as CTNNB1 has been implicated in fibrotic disorders a dsRNA as described herein may be used to treat a patient having a fibrotic disorder that may benefit from inhibition of CTNNB1 expression such as liver fibrosis or pulmonary fibrosis, for example.

As used herein, “introducing into a cell” when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. It is, for example envisaged that the dsRNA molecules disclosed herein be administered to a subject in need of medical intervention. Such an administration may comprise the injection of the dsRNA into a diseased site in said subject, for example into liver tissue/cells or into cancerous tissues/cells, like liver cancer tissue. In addition, the injection is preferably in close proximity to the diseased tissue envisaged. In vitro introduction into a cell includes methods known in the art such as electroporation and lipofection.

The dsRNA molecules described herein may be utilized in pharmaceutical compositions by adding an effective amount of the molecule to a suitable pharmaceutically acceptable diluent or carrier. Use of the dsRNAs and methods described herein may be useful prophylactically, e.g., to prevent or delay infection, inflammation or tumor formation, for example.

When delivered to animals, such as for treatment or prophylaxis, the dsRNAs are preferably non-immunostimulatory. The term “non-immunostimulatory” as used herein refers to the absence of induction of an immune response by the invented dsRNA molecules. Methods to determine immune responses are well known to a person skilled in the art, for example by assessing the release of cytokines, as described in the Examples section.

The dsRNAs described herein may be useful for research and diagnostic purposes, because these compounds hybridize to nucleic acids encoding CTNNB1, enabling sandwich and other assays to easily be constructed to exploit this fact. Hybridization of the dsRNAs with a nucleic acid encoding CTNNB1 can be detected by means known in the art. Such means may include conjugation of an enzyme to the dsRNA, radiolabelling of the dsRNA or any other suitable detection means. Kits using such detection means for detecting the level of CTNNB1 in a sample may also be prepared.

The dsRNAs described herein may be included in pharmaceutical compositions and formulations. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for parenteral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions containing a dsRNA described herein include, but are not limited to, solutions, emulsions, micellular formulations, and liposome-containing formulations. These compositions may be generated from a variety of components that include, but are not limited to, preformed liquids, self-emulsifying solids and self-emulsifying semisolids.

Pharmaceutical compositions containing a dsRNA described herein, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Compositions containing a dsRNA as described herein may be formulated into any of many possible dosage forms such as, but not limited to, tablets, capsules, liquid syrups, soft gels, suppositories, and enemas. The compositions may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Compositions containing a dsRNA as described herein may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, and may be given once or more daily, weekly, monthly or yearly, or even once every 2 to 20 years. Further examples of suitable dosages include 0.25 μg to 10 μg and 1 μg to 5 μg per kg of body weight. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

A number of embodiments of dsRNAs, compositions and methods are described herein. A summary of selected aspects of such compositions and methods is provided below.

In first aspect, a dsRNA molecule includes a duplex region comprising a sense region and an antisense region at least substantially complementary to the sense region. The sense region and the antisense region each comprise between 18 and 30 nucleotides. The antisense region comprises a nucleotide sequence that is fully complementary to at least 15 contiguous nucleotides of any one of SEQ ID NOs:1-24.

A second aspect is a dsRNA molecule according to the first aspect, wherein the antisense region comprises a nucleotide sequence that is fully complementary to at least 18 contiguous nucleotides of any one of SEQ ID NOs:1-24.

A third aspect is a dsRNA molecule according to the first aspect, wherein the antisense region consists a nucleotide sequence that is fully complementary to any one of SEQ ID NOs:1-24.

A fourth aspect is a dsRNA molecule according to the first aspect, wherein the antisense region comprises a nucleotide sequence that is fully complementary to at least 15 contiguous nucleotides of any one of SEQ ID NOs: 1-6.

A fifth aspect is a dsRNA molecule according to the first aspect, wherein the antisense region consists of a nucleotide sequence that is fully complementary to any one of SEQ ID NOs:1-6.

A sixth aspect is a dsRNA molecule according to any one of the preceding aspects, wherein one or more of the nucleotides of the antisense region are modified at the 2′ position of the ribose moiety.

A seventh aspect is a dsRNA molecule according to any one of the preceding aspects, wherein all the nucleotides of the antisense region are modified at the 2′ position of the ribose moiety.

An eighth aspect is a dsRNA molecule according to the sixth or seventh aspects, wherein the 2′ position of ribose moiety is substituted with fluoro or methoxy.

A ninth aspect is a dsRNA molecule according to any of the preceding aspects, wherein the antisense region comprises at least 15 contiguous nucleotides any one of SEQ ID NOs: 73-80.

A tenth aspect is a dsRNA molecule according to any of the preceding aspects, wherein the antisense region consists of any one of SEQ ID NOs: 73-80.

An eleventh aspect is a dsRNA molecule according to the first aspect, (i) wherein the sense region consists of a nucleotide sequence of nucleotides 1-19 of SEQ ID NO:49 and the antisense region consists of a nucleotide sequence of nucleotides 1-19 of SEQ ID NO:73, (ii) wherein the sense region consists of a nucleotide sequence of nucleotides 1-19 of SEQ ID NO:50 and the antisense region consists of a nucleotide sequence of nucleotides 1-19 of SEQ ID NO:74, (iii) wherein the sense region consists of a nucleotide sequence of nucleotides 1-19 of SEQ ID NO:51 and the antisense region consists of a nucleotide sequence of nucleotides 1-19 of SEQ ID NO:75, (iv) wherein the sense region consists of a nucleotide sequence of nucleotides 1-19 of SEQ ID NO:52 and the antisense region consists of a nucleotide sequence of nucleotides 1-19 of SEQ ID NO:76, (v) wherein the sense region consists of a nucleotide sequence of nucleotides 1-19 of SEQ ID NO:53 and the antisense region consists of a nucleotide sequence of nucleotides 1-19 of SEQ ID NO:77, (vi) wherein the sense region consists of a nucleotide sequence of nucleotides 1-19 of SEQ ID NO:54 and the antisense region consists of a nucleotide sequence of nucleotides 1-19 of SEQ ID NO:78, (vii) wherein the sense region consists of a nucleotide sequence of nucleotides 1-19 of SEQ ID NO:55 and the antisense region consists of a nucleotide sequence of nucleotides 1-19 of SEQ ID NO:79, (viii) wherein the sense region consists of a nucleotide sequence of nucleotides 1-19 of SEQ ID NO:56 and the antisense region consists of a nucleotide sequence of nucleotides 1-19 of SEQ ID NO:80, (ix) wherein the sense region consists of a nucleotide sequence of nucleotides 1-19 of SEQ ID NO:57 and the antisense region consists of a nucleotide sequence of nucleotides 1-19 of SEQ ID NO:81, (x) wherein the sense region consists of a nucleotide sequence of nucleotides 1-19 of SEQ ID NO:58 and the antisense region consists of a nucleotide sequence of nucleotides 1-19 of SEQ ID NO:82, (xi) wherein the sense region consists of a nucleotide sequence of nucleotides 1-19 of SEQ ID NO:59 and the antisense region consists of a nucleotide sequence of nucleotides 1-19 of SEQ ID NO:83, (xii) wherein the sense region consists of a nucleotide sequence of nucleotides 1-19 of SEQ ID NO:60 and the antisense region consists of a nucleotide sequence of nucleotides 1-19 of SEQ ID NO:84, (xiii) wherein the sense region consists of a nucleotide sequence of nucleotides 1-19 of SEQ ID NO:61 and the antisense region consists of a nucleotide sequence of nucleotides 1-19 of SEQ ID NO:85, (xiv) wherein the sense region consists of a nucleotide sequence of nucleotides 1-19 of SEQ ID NO:62 and the antisense region consists of a nucleotide sequence of nucleotides 1-19 of SEQ ID NO:86, (xv) wherein the sense region consists of a nucleotide sequence of nucleotides 1-19 of SEQ ID NO:63 and the antisense region consists of a nucleotide sequence of nucleotides 1-19 of SEQ ID NO:87, (xvi) wherein the sense region consists of a nucleotide sequence of nucleotides 1-19 of SEQ ID NO:64 and the antisense region consists of a nucleotide sequence of nucleotides 1-19 of SEQ ID NO:88, (xvii) wherein the sense region consists of a nucleotide sequence of nucleotides 1-19 of SEQ ID NO:65 and the antisense region consists of a nucleotide sequence of nucleotides 1-19 of SEQ ID NO:89, (xviii) wherein the sense region consists of a nucleotide sequence of nucleotides 1-19 of SEQ ID NO:66 and the antisense region consists of a nucleotide sequence of nucleotides 1-19 of SEQ ID NO:90, (xix) wherein the sense region consists of a nucleotide sequence of nucleotides 1-19 of SEQ ID NO:67 and the antisense region consists of a nucleotide sequence of nucleotides 1-19 of SEQ ID NO:91, (xx) wherein the sense region consists of a nucleotide sequence of nucleotides 1-19 of SEQ ID NO:68 and the antisense region consists of a nucleotide sequence of nucleotides 1-19 of SEQ ID NO:92, (xxi) wherein the sense region consists of a nucleotide sequence of nucleotides 1-19 of SEQ ID NO:69 and the antisense region consists of a nucleotide sequence of nucleotides 1-19 of SEQ ID NO:93, (xxii) wherein the sense region consists of a nucleotide sequence of nucleotides 1-19 of SEQ ID NO:70 and the antisense region consists of a nucleotide sequence of nucleotides 1-19 of SEQ ID NO:94, (xxiii) wherein the sense region consists of a nucleotide sequence of nucleotides 1-19 of SEQ ID NO:71 and the antisense region consists of a nucleotide sequence of nucleotides 1-19 of SEQ ID NO:95, or (xxiv) wherein the sense region consists of a nucleotide sequence of nucleotides 1-19 of SEQ ID NO:72 and the antisense region consists of a nucleotide sequence of nucleotides 1-19 of SEQ ID NO:96.

A twelfth aspect is a dsRNA molecule according to the first aspect, (i) wherein the sense region consists of a nucleotide sequence of nucleotides 1-19 of SEQ ID NO:49 and the antisense region consists of a nucleotide sequence of nucleotides 1-19 of SEQ ID NO:73, (ii) wherein the sense region consists of a nucleotide sequence of nucleotides 1-19 of SEQ ID NO:50 and the antisense region consists of a nucleotide sequence of nucleotides 1-19 of SEQ ID NO:74, (iii) wherein the sense region consists of a nucleotide sequence of nucleotides 1-19 of SEQ ID NO:51 and the antisense region consists of a nucleotide sequence of nucleotides 1-19 of SEQ ID NO:75, (iv) wherein the sense region consists of a nucleotide sequence of nucleotides 1-19 of SEQ ID NO:52 and the antisense region consists of a nucleotide sequence of nucleotides 1-19 of SEQ ID NO:76; (v) wherein the sense region consists of a nucleotide sequence of nucleotides 1-19 of SEQ ID NO:53 and the antisense region consists of a nucleotide sequence of nucleotides 1-19 of SEQ ID NO:77; (vi) wherein the sense region consists of a nucleotide sequence of nucleotides 1-19 of SEQ ID NO:54 and the antisense region consists of a nucleotide sequence of nucleotides 1-19 of SEQ ID NO:78; (vii) wherein the sense region consists of a nucleotide sequence of nucleotides 1-19 of SEQ ID NO:55 and the antisense region consists of a nucleotide sequence of nucleotides 1-19 of SEQ ID NO:79; or (viii) wherein the sense region consists of a nucleotide sequence of nucleotides 1-19 of SEQ ID NO:56 and the antisense region consists of a nucleotide sequence of nucleotides 1-19 of SEQ ID NO:80.

A thirteenth aspect is a dsRNA molecule according to any of the preceding aspects, further comprising one or two overhang regions, each comprising five or fewer nucleotides.

A fourteenth aspect is a dsRNA molecule according to the thirteenth aspect, wherein the one or two overhang regions comprise deoxythymidine (dT).

A fifteenth aspect is a dsRNA molecule according to the thirteenth aspect, wherein the one or two overhang regions comprise dT-phosphorothioate-dT.

A sixteenth aspect is a dsRNA molecule according to the thirteenth aspect, wherein the one or two overhang regions consist of dT-phosphorothioate-dT.

A seventeenth aspect is a dsRNA molecule according to the first aspect, wherein the dsRNA consists of: (i) a first nucleotide strand comprising the sense region, wherein the first nucleotide strand consists of a nucleotide of SEQ ID NO:49 and a second nucleotide strand comprising the antisense strand, wherein the second nucleotide strand consists of a nucleotide of SEQ ID NO:73; (ii) a first nucleotide strand comprising the sense region, wherein the first nucleotide strand consists of a nucleotide of SEQ ID NO:50 and a second nucleotide strand comprising the antisense strand, wherein the second nucleotide strand consists of a nucleotide of SEQ ID NO:74; (iii) a first nucleotide strand comprising the sense region, wherein the first nucleotide strand consists of a nucleotide of SEQ ID NO:51 and a second nucleotide strand comprising the antisense strand, wherein the second nucleotide strand consists of a nucleotide of SEQ ID NO:75; (iv) a first nucleotide strand comprising the sense region, wherein the first nucleotide strand consists of a nucleotide of SEQ ID NO:52 and a second nucleotide strand comprising the antisense strand, wherein the second nucleotide strand consists of a nucleotide of SEQ ID NO:76; (v) a first nucleotide strand comprising the sense region, wherein the first nucleotide strand consists of a nucleotide of SEQ ID NO:53 and a second nucleotide strand comprising the antisense strand, wherein the second nucleotide strand consists of a nucleotide of SEQ ID NO:77; (vi) a first nucleotide strand comprising the sense region, wherein the first nucleotide strand consists of a nucleotide of SEQ ID NO:54 and a second nucleotide strand comprising the antisense strand, wherein the second nucleotide strand consists of a nucleotide of SEQ ID NO:78; (vii) a first nucleotide strand comprising the sense region, wherein the first nucleotide strand consists of a nucleotide of SEQ ID NO:55 and a second nucleotide strand comprising the antisense strand, wherein the second nucleotide strand consists of a nucleotide of SEQ ID NO:79; (viii) a first nucleotide strand comprising the sense region, wherein the first nucleotide strand consists of a nucleotide of SEQ ID NO:56 and a second nucleotide strand comprising the antisense strand, wherein the second nucleotide strand consists of a nucleotide of SEQ ID NO:80; (ix) a first nucleotide strand comprising the sense region, wherein the first nucleotide strand consists of a nucleotide of SEQ ID NO:57 and a second nucleotide strand comprising the antisense strand, wherein the second nucleotide strand consists of a nucleotide of SEQ ID NO:81; (x) a first nucleotide strand comprising the sense region, wherein the first nucleotide strand consists of a nucleotide of SEQ ID NO:58 and a second nucleotide strand comprising the antisense strand, wherein the second nucleotide strand consists of a nucleotide of SEQ ID NO:82; (xi) a first nucleotide strand comprising the sense region, wherein the first nucleotide strand consists of a nucleotide of SEQ ID NO:59 and a second nucleotide strand comprising the antisense strand, wherein the second nucleotide strand consists of a nucleotide of SEQ ID NO:83; (xii) a first nucleotide strand comprising the sense region, wherein the first nucleotide strand consists of a nucleotide of SEQ ID NO:60 and a second nucleotide strand comprising the antisense strand, wherein the second nucleotide strand consists of a nucleotide of SEQ ID NO:84; (xiii) a first nucleotide strand comprising the sense region, wherein the first nucleotide strand consists of a nucleotide of SEQ ID NO:61 and a second nucleotide strand comprising the antisense strand, wherein the second nucleotide strand consists of a nucleotide of SEQ ID NO:85; (xiv) a first nucleotide strand comprising the sense region, wherein the first nucleotide strand consists of a nucleotide of SEQ ID NO:62 and a second nucleotide strand comprising the antisense strand, wherein the second nucleotide strand consists of a nucleotide of SEQ ID NO:86; (xv) a first nucleotide strand comprising the sense region, wherein the first nucleotide strand consists of a nucleotide of SEQ ID NO:63 and a second nucleotide strand comprising the antisense strand, wherein the second nucleotide strand consists of a nucleotide of SEQ ID NO:87; (xvi) a first nucleotide strand comprising the sense region, wherein the first nucleotide strand consists of a nucleotide of SEQ ID NO:64 and a second nucleotide strand comprising the antisense strand, wherein the second nucleotide strand consists of a nucleotide of SEQ ID NO:88; (xvii) a first nucleotide strand comprising the sense region, wherein the first nucleotide strand consists of a nucleotide of SEQ ID NO:65 and a second nucleotide strand comprising the antisense strand, wherein the second nucleotide strand consists of a nucleotide of SEQ ID NO:89; (xviii) a first nucleotide strand comprising the sense region, wherein the first nucleotide strand consists of a nucleotide of SEQ ID NO:66 and a second nucleotide strand comprising the antisense strand, wherein the second nucleotide strand consists of a nucleotide of SEQ ID NO:90; (xix) a first nucleotide strand comprising the sense region, wherein the first nucleotide strand consists of a nucleotide of SEQ ID NO:67 and a second nucleotide strand comprising the antisense strand, wherein the second nucleotide strand consists of a nucleotide of SEQ ID NO:91; (xx) a first nucleotide strand comprising the sense region, wherein the first nucleotide strand consists of a nucleotide of SEQ ID NO:68 and a second nucleotide strand comprising the antisense strand, wherein the second nucleotide strand consists of a nucleotide of SEQ ID NO:92; (xxi) a first nucleotide strand comprising the sense region, wherein the first nucleotide strand consists of a nucleotide of SEQ ID NO:69 and a second nucleotide strand comprising the antisense strand, wherein the second nucleotide strand consists of a nucleotide of SEQ ID NO:93; (xxii) a first nucleotide strand comprising the sense region, wherein the first nucleotide strand consists of a nucleotide of SEQ ID NO:70 and a second nucleotide strand comprising the antisense strand, wherein the second nucleotide strand consists of a nucleotide of SEQ ID NO:94; (xxiii) a first nucleotide strand comprising the sense region, wherein the first nucleotide strand consists of a nucleotide of SEQ ID NO:71 and a second nucleotide strand comprising the antisense strand, wherein the second nucleotide strand consists of a nucleotide of SEQ ID NO:95; or (xxiv) a first nucleotide strand comprising the sense region, wherein the first nucleotide strand consists of a nucleotide of SEQ ID NO:72 and a second nucleotide strand comprising the antisense strand, wherein the second nucleotide strand consists of a nucleotide of SEQ ID NO:96.

An eighteenth aspect is a dsRNA molecule according the first aspect, wherein the dsRNA consists of (i) a first nucleotide strand comprising the sense region, wherein the first nucleotide strand consists of a nucleotide of SEQ ID NO:49 and a second nucleotide strand comprising the antisense strand, wherein the second nucleotide strand consists of a nucleotide of SEQ ID NO:73; (ii) a first nucleotide strand comprising the sense region, wherein the first nucleotide strand consists of a nucleotide of SEQ ID NO:50 and a second nucleotide strand comprising the antisense strand, wherein the second nucleotide strand consists of a nucleotide of SEQ ID NO:74; (iii) a first nucleotide strand comprising the sense region, wherein the first nucleotide strand consists of a nucleotide of SEQ ID NO:51 and a second nucleotide strand comprising the antisense strand, wherein the second nucleotide strand consists of a nucleotide of SEQ ID NO:75; (iv) a first nucleotide strand comprising the sense region, wherein the first nucleotide strand consists of a nucleotide of SEQ ID NO:52 and a second nucleotide strand comprising the antisense strand, wherein the second nucleotide strand consists of a nucleotide of SEQ ID NO:76; (v) a first nucleotide strand comprising the sense region, wherein the first nucleotide strand consists of a nucleotide of SEQ ID NO:53 and a second nucleotide strand comprising the antisense strand, wherein the second nucleotide strand consists of a nucleotide of SEQ ID NO:77; (vi) a first nucleotide strand comprising the sense region, wherein the first nucleotide strand consists of a nucleotide of SEQ ID NO:54 and a second nucleotide strand comprising the antisense strand, wherein the second nucleotide strand consists of a nucleotide of SEQ ID NO:78; (vii) a first nucleotide strand comprising the sense region, wherein the first nucleotide strand consists of a nucleotide of SEQ ID NO:55 and a second nucleotide strand comprising the antisense strand, wherein the second nucleotide strand consists of a nucleotide of SEQ ID NO:79; or (viii) a first nucleotide strand comprising the sense region, wherein the first nucleotide strand consists of a nucleotide of SEQ ID NO:56 and a second nucleotide strand comprising the antisense strand, wherein the second nucleotide strand consists of a nucleotide of SEQ ID NO:80.

A nineteenth aspect is a method for inhibiting expression of beta catenin in a cell. The method includes introducing into the cell a dsRNA molecule according to any one of the preceding aspects.

A twentieth aspect is a pharmaceutically acceptable composition comprising a dsRNA molecule according to any of aspects 1-18.

A twenty-first aspect is a method for inhibiting expression of beta catenin in a cell of a subject in need thereof. The method includes introducing into the cell of the subject a composition according to the twentieth aspect.

A twenty-second aspect is a method for treating cancer in a subject. The method includes administering to the subject a composition according to the twentieth aspect.

A twenty-third aspect is a method according to the twenty-second aspect, wherein the cancer is selected from the group consisting of basal cell carcinoma, colorectal cancer, pilomatrixoma, medullablastoma, liver cancer and ovarian cancer.

A twenty-fourth aspect is a method according to the twenty-second aspect, wherein the cancer is liver cancer selected from the group consisting of hepatocellular carcinoma and cholangiocarcinoma.

A twenty-fifth aspect is a method for treating fibrotic disorders in a subject. The method includes administering to the subject a composition according to the twentieth aspect.

A twenty-sixth aspect is a method according to the twenty-fifth aspect, wherein the fibrotic disorder is liver fibrosis or pulmonary fibrosis.

In the following, non-limiting examples are presented, which describe various embodiments of representative dsRNAs, compositions and methods inhibiting expression of CTNNB1.

### EXAMPLES

### Example 1

### Initial Identification of dsRNAs

dsRNA design was carried out to identify specific dsRNAs targeting human CTNNB1 for therapeutic use. First, known mRNA sequences of human (Homo sapiens) CTNNB1 (hs NM_{—}001904.3 listed as SEQ ID NO. 97, NM_{—}001098209.1 listed as SEQ ID NO. 98, and NM_{—}001098210.1 listed as SEQ ID NO. 99), the known mRNA sequences of rhesus monkey (Macaca mulatta) CTNNB1 (XM_{—}001115474.2 listed as SEQ ID NO. 100, XM_{—}002802838.1 listed as SEQ ID NO. 101, XM_{—}002802839.1 listed as SEQ ID NO. 102, XM_{—}002802840.1 listed as SEQ ID NO. 103 and XM_{—}002802841.1 listed as SEQ ID NO. 104) and the known mRNA sequences of mouse (Mus musculus) CTNNB1 (NM_{—}007614.3 listed as SEQ ID NO. 105 and NM_{—}001165902.1 listed as SEQ ID NO. 106) were downloaded from NCBI Genbank.

The human CTNNB1 mRNA sequences (SEQ ID NO. 97, SEQ ID NO. 98 and SEQ ID NO. 99) were examined together with the rhesus monkey CTNNB1 mRNA sequences (SEQ ID NO. 100, SEQ ID NO. 101, SEQ ID NO. 102, SEQ ID NO. 103 and SEQ ID NO. 104) and the mouse CTNNB1 mRNA sequences (SEQ ID NO. 105 and SEQ ID NO. 106) by computer analysis to identify homologous sequences of 19 nucleotides that yield RNA interference (RNAi) agents cross-reactive to all listed sequences.

Selection of 19mer sequences was restricted to the region from nucleotide position 269 to 2615 of human CTNNB1 mRNA (SEQ ID NO. 97).

From this initial set of sequences those harbouring a SNP (single nucleotide polymorphism) in their corresponding target sequence in human CTNNB1 mRNA (SEQ ID NO. 97) as indicated by the NCBI dbSNP (build 135) were excluded.

In identifying RNAi agents, the selection was limited to 19mer antisense sequences having at least 2 mismatches to any other sequence in the human and mouse NCBI RefSeq databases (release 52) and to 19mer sense sequences having at least 1 mismatch to any other sequence in the human and mouse NCBI RefSeq databases (release 52), which we assumed to represent the comprehensive human and mouse transcriptomes.

Selection of candidates was further limited by elimination of 19mer antisense strands harbouring seed sequences (nucleotides 2-7 of the 5′ terminus) identical to known human miRNA seed sequences (nucleotides 2-7 of the 5′ terminus). The candidate selection was additionally limited by elimination of 19mer sense strands harbouring seed sequences (nucleotides 2-7 of the 5′ terminus) identical to known miRNA seed sequences (nucleotides 2-7 of the 5′ terminus) that are conserved in human, rhesus monkey, mouse and rat (Rattus norvegicus) as listed in miRBase (University of Manchester, release 18).

In addition all sense and antisense sequences containing five or more consecutive G's (poly-G sequences) were excluded from the synthesis.

The sequences identified are presented in Table 1 below.

TABLE 1
Core sequences of double stranded RNAs
(dsRNAs) targeting human CTNNB1 gene.

| SEQ | Sense strand | SEQ | Antisense strand |
|---|---|---|---|
| ID | core sequence | ID | core sequence |
| NO | (5′-3′) | NO | (5′-3′) |
| 1 | CAGGGGUCCUCUGUGAACU | 25 | AGUUCACAGAGGACCCCUG |
| 2 | UGCUCUUCGUCAUCUGACC | 26 | GGUCAGAUGACGAAGAGCA |
| 3 | GCUCUUCGUCAUCUGACCA | 27 | UGGUCAGAUGACGAAGAGC |
| 4 | GGAGCUAAAAUGGCAGUGC | 28 | GCACUGCCAUUUUAGCUCC |
| 5 | CCUGUGCAGCUGGAAUUCU | 29 | AGAAUUCCAGCUGCACAGG |
| 6 | AGAGUAGCUGCAGGGGUCC | 30 | GGACCCCUGCAGCUACUCU |
| 7 | CUGACUAUCCAGUUGAUGG | 31 | CCAUCAACUGGAUAGUCAG |
| 8 | CCAUUCCAUUGUUUGUGCA | 32 | UGCACAAACAAUGGAAUGG |
| 9 | AUACCAUUCCAUUGUUUGU | 33 | ACAAACAAUGGAAUGGUAU |
| 10 | GCAGGGGUCCUCUGUGAAC | 34 | GUUCACAGAGGACCCCUGC |
| 11 | CCAGGACCUCAUGGAUGGG | 35 | CCCAUCCAUGAGGUCCUGG |
| 12 | UACCAUUCCAUUGUUUGUG | 36 | CACAAACAAUGGAAUGGUA |
| 13 | UGUGAACUUGCUCAGGACA | 37 | UGUCCUGAGCAAGUUCACA |
| 14 | UGGAUAUCGCCAGGAUGAU | 38 | AUCAUCCUGGCGAUAUCCA |
| 15 | UGACUAUCCAGUUGAUGGG | 39 | CCCAUCAACUGGAUAGUCA |
| 16 | ACCAUGCAGAAUACAAAUG | 40 | CAUUUGUAUUCUGCAUGGU |
| 17 | ACUGUUGGAUUGAUUCGAA | 41 | UUCGAAUCAAUCCAACAGU |
| 18 | CUAUCCAGUUGAUGGGCUG | 42 | CAGCCCAUCAACUGGAUAG |
| 19 | GACUAUCCAGUUGAUGGGC | 43 | GCCCAUCAACUGGAUAGUC |
| 20 | GCUGACUAUCCAGUUGAUG | 44 | CAUCAACUGGAUAGUCAGC |
| 21 | AAUACCAUUCCAUUGUUUG | 45 | CAAACAAUGGAAUGGUAUU |
| 22 | ACCCUGGUGCUGACUAUCC | 46 | GGAUAGUCAGCACCAGGGU |
| 23 | UGCUUUAUUCUCCCAUUGA | 47 | UCAAUGGGAGAAUAAAGCA |
| 24 | AGGAGCUAAAAUGGCAGUG | 48 | CACUGCCAUUUUAGCUCCU |

In Table 1, letters in capitals represent RNA nucleotides. Core sense strand and core antisense strand form the core double stranded region of a dsRNA molecule. In Table 1, a dsRNA pair is shown within a row. For example, the SEQ ID NO:1 sense strand and the SEQ ID NO:25 antisense strand form a dsRNA, the SEQ ID NO:2 sense strand and the SEQ ID NO:26 antisense strand form a dsRNA, SEQ ID NO:3 sense strand and the SEQ ID NO:27 antisense strand form a dsRNA, and so on.

### Example 2

### dsRNA Synthesis

Where the source of a reagent is not specifically given herein, such reagent may be obtained from any supplier of reagents for molecular biology at a quality/purity standard for application in molecular biology.

Oligoribonucleotides were synthesized according to the phosphoramidite technology on solid phase at a scale of 0.2 μmol employing an ABI3900 synthesizer (ABI Biosystems). Synthesis was performed on solid supports made of polystyrene obtained from Glen Research. RNA phosphoramidites, (5′-O-dimethoxytrityl-N6-(benzoyl)-2′-O-t-butyldimethylsilyl-adenosine-3′-O-(2-cyanoethyl-N,N-diisopropylamino) phosphoramidite, 5′-O-dimethoxytrityl-N4-(acetyl)-2′-O-t-butyldimethylsilyl-cytidine-3′-O-(2-cyanoethyl-N,N-diisopropylamino) phosphoramidite, (5′-O-dimethoxytrityl-N2-(isobutyl)-2′-O-t-butyldimethylsilyl-guanosine-3′-O-(2-cyanoethyl-N,N-diisopropylamino) phosphoramidite, and 5′-O-dimethoxytrityl-2′-O-t-butyldimethylsilyl-uridine-3′-O-(2-cyanoethyl-N,N-diisopropylamino) phosphoramidite were purchased from SAFC Proligo. 2′-O-Methylphosphoramidites as well as 2′-deoxy-2′-fluorophosphoramidites (SAFC Proligo) carried the same protecting groups as the regular amidites. All amidites were dissolved in anhydrous acetonitrile (70 mM) and molecular sieves (3\acute{Å}) were added. 5-ethyl thiotetrazole (ETT, 500 mM in acetonitrile) was used as activator solution. Coupling times were 4 minutes. Oxidation was carried out either with a mixture of iodine/water/pyridine (50 mM/10%/90% (v/v)) or by 50 mM DDTT (AM Chemicals) in pyridine/ACN (50/50 v/v) in order to introduce phosphorothioate linkages. Standard capping reagents were used. The RNA building blocks were incorporated within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA.

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleiβheim, Germany). Double stranded RNA was generated by mixing an equimolar solution of complementary strands in annealing buffer (20 mM sodium phosphate, pH 6.8; 100 mM sodium chloride), heated in a water bath at 70° C. for 3 minutes and cooled to room temperature over a period of 3-4 hours. The annealed RNA solution was stored at −20° C. until use.

The dsRNAs synthesized are presented in Table 2 below.

TABLE 2
Synthesized dsRNAs.

| SEQ | | SEQ | |
|---|---|---|---|
| ID | Sense strand | ID | Antisense strand |
| NO | sequence (5′-3′) | NO | sequence (5′-3′) |
| 49 | caGfgGfgUfcCfuCfu | 73 | AfGfuUfcAfcAfgAfg |
| | GfuGfaAfcUfdTsdT | | GfaCfcCfcUfgdTsdT |
| 50 | ugCfuCfuUfcGfuCfa | 74 | GfGfuCfaGfaUfgAfc |
| | UfcUfgAfcCfdTsdT | | GfaAfgAfgCfadTsdT |
| 51 | gcUfcUfuCfgUfcAfu | 75 | UfGfgUfcAfgAfuGfa |
| | CfuGfaCfcAfdTsdT | | CfgAfaGfaGfcdTsdT |
| 52 | ggAfgCfuAfaAfaUfg | 76 | GfCfaCfuGfcCfaUfu |
| | GfcAfgUfgCfdTsdT | | UfuAfgCfuCfcdTsdT |
| 53 | ccUfgUfgCfaGfcUfg | 77 | AfGfaAfuUfcCfaGfc |
| | GfaAfuUfcUfdTsdT | | UfgCfaCfaGfgdTsdT |
| 54 | agAfgUfaGfcUfgCfa | 78 | GfGfaCfcCfcUfgCfa |
| | GfgGfgUfcCfdTsdT | | GfcUfaCfuCfudTsdT |
| 55 | cuGfaCfuAfuCfcAfg | 79 | CfCfaUfcAfaCfuGfg |
| | UfuGfaUfgGfdTsdT | | AfuAfgUfcAfgdTsdT |
| 56 | ccAfuUfcCfaUfuGfu | 80 | UfGfcAfcAfaAfcAfa |
| | UfuGfuGfcAfdTsdT | | UfgGfaAfuGfgdTsdT |
| 57 | auAfcCfaUfuCfcAfu | 81 | AfCfaAfaCfaAfuGfg |
| | UfgUfuUfgUfdTsdT | | AfaUfgGfuAfudTsdT |
| 58 | gcAfgGfgGfuCfcUfc | 82 | GfUfuCfaCfaGfaGfg |
| | UfgUfgAfaCfdTsdT | | AfcCfcCfuGfcdTsdT |
| 59 | ccAfgGfaCfcUfcAfu | 83 | CfCfcAfuCfcAfuGfa |
| | GfgAfuGfgGfdTsdT | | GfgUfcCfuGfgdTsdT |
| 60 | uaCfcAfuUfcCfaUfu | 84 | CfAfcAfaAfcAfaUfg |
| | GfuUfuGfuGfdTsdT | | GfaAfuGfgUfadTsdT |
| 61 | ugUfgAfaCfuUfgCfu | 85 | UfGfuCfcUfgAfgCfa |
| | CfaGfgAfcAfdTsdT | | AfgUfuCfaCfadTsdT |
| 62 | ugGfaUfaUfcGfcCfa | 86 | AfUfcAfuCfcUfgGfc |
| | GfgAfuGfaUfdTsdT | | GfaUfaUfcCfadTsdT |
| 63 | ugAfcUfaUfcCfaGfu | 87 | CfCfcAfuCfaAfcUfg |
| | UfgAfuGfgGfdTsdT | | GfaUfaGfuCfadTsdT |
| 64 | acCfaUfgCfaGfaAfu | 88 | CfAfuUfuGfuAfuUfc |
| | AfcAfaAfuGfdTsdT | | UfgCfaUfgGfudTsdT |
| 65 | acUfgUfuGfgAfuUfg | 89 | UfUfcGfaAfuCfaAfu |
| | AfuUfcGfaAfdTsdT | | CfcAfaCfaGfudTsdT |
| 66 | cuAfuCfcAfgUfuGfa | 90 | CfAfgCfcCfaUfcAfa |
| | UfgGfgCfuGfdTsdT | | CfuGfgAfuAfgdTsdT |
| 67 | gaCfuAfuCfcAfgUfu | 91 | GfCfcCfaUfcAfaCfu |
| | GfaUfgGfgCfdTsdT | | GfgAfuAfgUfcdTsdT |
| 68 | gcUfgAfcUfaUfcCfa | 92 | CfAfuCfaAfcUfgGfa |
| | GfuUfgAfuGfdTsdT | | UfaGfuCfaGfcdTsdT |
| 69 | aaUfaCfcAfuUfcCfa | 93 | CfAfaAfcAfaUfgGfa |
| | UfuGfuUfuGfdTsdT | | AfuGfgUfaUfudTsdT |
| 70 | acCfcUfgGfuGfcUfg | 94 | GfGfaUfaGfuCfaGfc |
| | AfcUfaUfcCfdTsdT | | AfcCfaGfgGfudTsdT |
| 71 | ugCfuUfuAfuUfcUfc | 95 | UfCfaAfuGfgGfaGfa |
| | CfcAfuUfgAfdTsdT | | AfuAfaAfgCfadTsdT |
| 72 | agGfaGfcUfaAfaAfu | 96 | CfAfcUfgCfcAfuUfu |
| | GfgCfaGfuGfdTsdT | | UfaGfcUfcCfudTsdT |

In Table 2, letters in capitals represent RNA nucleotides, lower case letters “c”, “g”, “a” and “u” represent 2′-O-methyl-modified nucleotides, “s” represents phosphorothioate and “dT” represents deoxythymidine residues. Upper case letters A, C, G, U followed by “f” indicate 2′-fluoro nucleotides. The modified dsRNAs presented in Table 2 correspond to the unmodified dsRNAs presented in Table 1, as follows: SEQ ID NO: 49 corresponds to SEQ ID NO:1, SEQ ID NO:50 corresponds to SEQ ID NO:2, SEQ ID NO:51 corresponds to SEQ ID NO:3, and so on.

In Table 2, a dsRNA pair is shown within a row. For example, the SEQ ID NO:49 sense strand and the SEQ ID NO:73 antisense strand form a dsRNA, the SEQ ID NO:50 sense strand and the SEQ ID NO:74 antisense strand form a dsRNA, SEQ ID NO:51 sense strand and the SEQ ID NO:75 antisense strand form a dsRNA, and so on.

### Example 3

### Determination of dsRNA Activity

HeLa cells and HCT116 cells in culture were used for quantitation of CTNNB1 mRNA by branched DNA in total mRNA from cells transfected with CTNNB1 specific siRNAs.

HeLa cells were obtained from American Type Culture Collection (Rockville, Md., cat. No. CCL-2.2) and cultured in Ham's F12 medium (Biochrom AG, Berlin, Germany, cat. No. FG 0815) supplemented to contain 10% fetal calf serum (FCS) (Biochrom AG, Berlin, Germany, cat. No. 50115) and Penicillin 100 U/ml, Streptomycin 100 mg/ml (Biochrom AG, Berlin, Germany, cat. No. A2213) at 37° C. in an atmosphere with 5% CO₂ in a humidified incubator (Heraeus HERAcell, Kendro Laboratory Products, Langenselbold, Germany).

HCT116 cells were obtained from Leibniz Institute DSMZ-German Collection of Microorganisms and Cell Culture (DSMZ, Braunschweig, Germany, cat. No. ACC-581) and cultured in McCoy's 5A medium (Biochrom AG, Berlin, Germany, cat. No. F1015) supplemented to contain 10% fetal calf serum (FCS) (Biochrom AG, Berlin, Germany, cat. No. S0115), 2 mM L-Glutamine (Biochrom AG, Berlin, Germany, cat. No. K0283), and Penicillin 100 U/ml, Streptomycin 100 mg/ml (Biochrom AG, Berlin, Germany, cat. No. A2213) at 37° C. in an atmosphere with 5% CO₂ in a humidified incubator (Heraeus HERAcell, Kendro Laboratory Products, Langenselbold, Germany).

Transfections of HeLa and HCT116 cells with dsRNA were performed directly after seeding 15000 cells/well on a 96-well plate, and were carried out with Lipofectamine2000 (Invitrogen GmbH, Karlsruhe, Germany, cat. No. 11668-019) essentially as described by the manufacturer.

In an initial single dose experiment performed in quadruplicates, HeLa cells were transfected with dsRNAs at a concentration of 50 nM and 5 nM. Most effective dsRNAs against CTNNB1 from the initial dose screen were further characterized by dose response curves in both HeLa and HCT116 cells. For dose response curves, transfections were performed as described above, but with dsRNA concentrations starting with 24 nM and decreasing in 4-fold dilutions down to 92 fM. After transfection, cells were incubated for 24 h at 37° C. and 5% CO₂ in a humidified incubator (Heraeus GmbH, Hanau, Germany). For measurement of CTNNB1 mRNA cells were harvested and lysed at 53° C. following procedures recommended by the manufacturer. For quantitation of GAPDH-mRNA the Quantigene Explore Kit (Panomics, Fremont, Calif., USA, cat. No. QG0004) was used, whereas quantitation of CTNNB1 mRNA was conducted with QuantiGene 2.0 (custom manufacturing for Axolabs GmbH, Kulmbach, Germany). After incubation and lysis, 50 μl of the lysates were incubated with probe-sets specific to human CTNNB1 and 10 μl of the lysates were incubated with probe-sets specific to human GAPDH. Both reaction types were processed according to the manufacturer's protocol for the respective QuantiGene kit. Chemoluminescence was measured in a Victor2-Light (Perkin Elmer, Wiesbaden, Germany) as RLUs (relative light units) and values obtained with the human CTNNB1 probe-set were normalized to the respective human GAPDH values for each well and then normalized to the corresponding mRNA readout from cells treated with three unrelated control siRNAs.

mRNA reduction data is presented in Table 3 and Table 4 below.

TABLE 3
Determination of activity for dsRNAs targeting human CTNNB1:
Activity was tested after transfection of HeLa cells with siRNA.
Activity is determined by quantitation of the remaining mRNA
normalized to control siRNA treated cells. Mean and standard
deviation are based on analysis of four replicates.

| | Remaining mRNA after | | Remaining mRNA after | |
|---|---|---|---|---|
| | transfection of HeLa cells | | transfection of HeLa cells | |
| | with 50 nM dsRNA | | with 5 nM dsRNA | |
| SEQ ID | mean | standard | mean | standard |
| NO PAIR | [%] | deviation [%] | [%] | deviation [%] |
| 49/73 | 7 | 0 | 6 | 1 |
| 50/74 | 8 | 1 | 6 | 1 |
| 51/75 | 7 | 1 | 6 | 1 |
| 52/76 | 9 | 1 | 7 | 2 |
| 53/77 | 16 | 1 | 7 | 1 |
| 54/78 | 8 | 1 | 7 | 1 |
| 55/79 | 8 | 0 | 7 | 1 |
| 56/80 | 8 | 0 | 8 | 0 |
| 57/81 | 9 | 0 | 8 | 1 |
| 58/82 | 27 | 3 | 8 | 1 |
| 59/83 | 10 | 1 | 8 | 2 |
| 60/84 | 12 | 1 | 9 | 1 |
| 61/85 | 12 | 1 | 10 | 2 |
| 62/86 | 13 | 1 | 10 | 2 |
| 63/87 | 12 | 1 | 11 | 2 |
| 64/88 | 21 | 1 | 11 | 2 |
| 65/89 | 13 | 1 | 13 | 2 |
| 66/90 | 16 | 0 | 13 | 1 |
| 67/91 | 19 | 1 | 15 | 2 |
| 68/92 | 31 | 5 | 17 | 3 |
| 69/93 | 15 | 1 | 19 | 3 |
| 70/94 | 41 | 3 | 35 | 6 |
| 71/95 | 35 | 1 | 37 | 3 |
| 72/96 | 79 | 13 | 70 | 19 |

For the results presented in Table 3, activity was tested after transfection of HeLa cells with siRNA. Activity is determined by quantitation of the remaining mRNA normalized to control siRNA treated cells. Mean and standard deviation are based on analysis of four replicates.

TABLE 4
Determination of dsRNA potency and efficacy.

| | Inhibitory concentrations and | | | | Inhibitory concentrations | | | |
|---|---|---|---|---|---|---|---|---|
| | maximal relative mRNA | | | | and maximal relative | | | |
| | reduction for selected | | | | mRNA reduction for | | | |
| | dsRNAs after | | | | selected dsRNAs after | | | |
| | transfection to HCT116 | | | | transfection to HeLa | | | |
| | cells, means of | | | | cells, means of four | | | |
| | four transfections | | | | transfections | | | |
| SEQ | | | | max. | | | | max. |
| ID | | | | mRNA | | | | mRNA |
| NO | IC50 | IC80 | IC20 | reduction | IC50 | IC80 | IC20 | reduction |
| pair | [nM] | [nM] | [nM] | [%] | [nM] | [nM] | [nM] | [%] |
| 49/73 | 0.020 | 0.170 | 0.002 | 94.8 | 0.007 | 0.056 | #N/A | 96.0 |
| 50/74 | 0.008 | 0.063 | 0.001 | 92.4 | 0.005 | 0.031 | 0.001 | 93.3 |
| 51/75 | 0.017 | 0.138 | 0.001 | 93.8 | 0.013 | 0.059 | 0.002 | 93.5 |
| 52/76 | 0.033 | 0.211 | 0.004 | 91.8 | 0.021 | 0.164 | 0.002 | 93.4 |
| 53/77 | 0.022 | 0.141 | 0.002 | 92.2 | 0.010 | 0.053 | 0.001 | 92.6 |
| 54/78 | 0.057 | 0.487 | 0.005 | 93.0 | 0.035 | 0.224 | 0.008 | 91.0 |
| 55/79 | 0.014 | 0.116 | 0.001 | 92.4 | 0.008 | 0.043 | 0.001 | 93.2 |
| 56/80 | 0.010 | 0.083 | 0.001 | 96.6 | 0.004 | 0.032 | #N/A | 94.2 |

Table 4 shows results of transfection of HeLa and HCT116 cells with selected dsRNA targeting human CTNNB1 in a dose response experiment. Data represent the mean of four transfection experiments. IC 50: 50% inhibitory concentration, IC 80: 80% inhibitory concentration, IC 20: 20% inhibitory concentration.

### Example 4

### Stability of dsRNAs

Stability of dsRNAs targeting human CTNNB1 was determined by incubation of the dsRNA for various times in human serum and subsequent analysis of the RNA strands by HPLC.

For each time point 3 μl 50 μM dsRNA sample was mixed with 30 μl human serum (Sigma). Mixtures were incubated for either 0 min, 30 min, 1 h, 3 h, 6 h, 24 h, or 48 h at 37° C. As a control for unspecific degradation 3 μl 50 μM dsRNA was incubated with 30 μl 1×PBS pH 7.4 for 48 h. Reactions were stopped by addition of proteinase K and incubation for 30 min at 65° C. Prior to sample analysis by HPLC, samples were diluted to a final volume of 200 μl with water.

For separation of single strands and analysis of remaining full length product (FLP), samples were analyzed by ion exchange chromatography on Dionex Summit HPLC under denaturing conditions. A gradient from 25% B to 62% B in 18 min was applied at a temperature of 50° C. using eluent A: 20 mM Na3PO4 in 10% ACN pH 11.0 and eluent B: 1M NaBr in eluent A.

For every sample, the chromatograms were integrated automatically by the Dionex Chromeleon 6.60 HPLC software and adjusted manually if necessary. All peak areas for the full length strand were normalized to the peak area of full length strand at t=0 min. The area under the peak and resulting remaining FLP was calculated for each single strand separately, when possible. For dsRNAs with coeluting single strands, the area under that peak was calculated and defined as remaining duplex.

Stability data are given in Table 5 below.

TABLE 5
Stability of dsRNAs targeting human CTNNB1.

| | Strand stability in human serum | | |
|---|---|---|---|
| SEQ ID | sense | antisense | dsRNA |
| NO pair | t½ [hr] | t½ [hr] | t½ [hr] |
| 49/73 | >48 | >48 | n.d. |
| 50/74 | >48 | >48 | n.d. |
| 51/75 | >48 | >48 | n.d. |
| 52/76 | >48 | >48 | n.d. |
| 53/77 | >48 | >48 | n.d. |
| 54/78 | >48 | >48 | n.d. |
| 55/79 | 48 | >48 | n.d. |
| 56/80 | n.d. | n.d. | >48 |

In Table 5, t½=half-life.

### Example 5

### Cytokine Induction

Potential cytokine induction of dsRNAs was determined by measuring the release of IFN-alpha and TNF-alpha from human peripheral blood mononuclear cells (PBMCs) after transfection with dsRNAs.

PBMCs were prepared from buffy coat blood (obtained from Institute of Transfusion Medicine, Suhl, Germany) of four donors by Ficoll (Sigma-Aldrich Chemie GmbH, Steinheim, Germany, cat. No. 10771) centrifugation prior to dsRNA treatment. Cells were transfected in quadruplicates with a final concentration of 133 nM dsRNA using either GenePorter2 (GP2) (Genlantis, San Diego, USA, cat. No. T202015) or DOTAP (Roche Diagnostics GmbH, Mannheim, Germany, cat. No. 11202375001) as transfection reagents. Cells were cultured for 24 h at 37° C. in Opti-MEM (Invitrogen/Life Technologies, Darmstadt, Germany, cat. No. 11058-021). dsRNA sequences known to induce IFN-alpha and TNF-alpha in this assay and CpG oligonucleotide, were used as positive controls.

At the end of incubation, IFN-alpha and TNF-alpha were measured in duplicates by standard sandwich ELISA (BenderMedSystems, Vienna, Austria, cat No. BMS216INSTCE for IFN-alpha and BenderMedSystems, Vienna, Austria, cat. No. BMS223INSTCE or R&D Systems, Wiesbaden-Nordenstadt, Germany, cat. No. DTA00C for TNF-alpha) from the cell culture supernatant.

The degree of cytokine induction was expressed relative to positive controls using a score from 1 to 5, with 5 indicating maximum induction.

Cytokine induction data are given in Table 6 below.

TABLE 6
Cytokine induction of dsRNAs targeting human CTNNB1.

Activation of Interferon-α and TNF-α
by dsRNA in human PBMC

| | | |
|---|---|---|
| SEQ ID | IFN-α | TNF-α |

| | |
|---|---|
| NO pair | [arbitrary units] |

| | | |
|---|---|---|
| 49/73 | 1 | 1 |
| 50/74 | 1 | 1 |
| 51/75 | 1 | 1 |
| 52/76 | 1 | 1 |
| 53/77 | 1 | 1 |
| 54/78 | 1 | 1 |
| 55/79 | 1 | 1 |
| 56/80 | 1 | 1 |

In Table 6, PBMC=peripheral blood mononuclear cells.

Thus, embodiments of RNA TARGETED TO BETA CATENIN are disclosed. One skilled in the art will appreciate that the compounds, compositions, and methods described herein can be practiced with embodiments other than those disclosed. The disclosed embodiments are presented for purposes of illustration and not limitation.

All publications, including but not limited to patents and patent applications, cited in this specification are herein incorporated by reference as if each individual publication were specifically and individually indicated to be incorporated by reference herein as though fully set forth.

1. A double stranded RNA (dsRNA) molecule comprising:
a duplex region comprising a sense region and an antisense region at least substantially complementary to the sense region,
wherein the sense region and the antisense region each comprise between 18 and 30 nucleotides, and
wherein the antisense region comprises a nucleotide sequence that is fully complementary to at least 15 contiguous nucleotides of any one of SEQ ID NOs: 1-24.

2. A dsRNA molecule according to claim 1, wherein the antisense region comprises a nucleotide sequence that is fully complementary to at least 18 contiguous nucleotides of any one of SEQ ID NOs: 1-24.

3. A dsRNA molecule according to claim 1, wherein the antisense region consists a nucleotide sequence that is fully complementary to any one of SEQ ID NOs: 1-24.

4. A dsRNA molecule according to claim 1, wherein the antisense region comprises a nucleotide sequence that is fully complementary to at least 15 contiguous nucleotides of any one of SEQ ID NOs: 1-6.

5. A dsRNA molecule according to claim 1, wherein the antisense region consists of a nucleotide sequence that is fully complementary to any one of SEQ ID NOs: 1-6.

6. A dsRNA molecule according to claim 1, wherein one or more of the nucleotides of the antisense region are modified at the 2′ position of the ribose moiety.

7. A dsRNA molecule according to claim 1, wherein all the nucleotides of the antisense region are modified at the 2′ position of the ribose moiety.

8. A dsRNA molecule according to claim 6, wherein the 2′ position of ribose moiety is substituted with fluoro or methoxy.

9. A dsRNA molecule according to claim 1, wherein the antisense region comprises at least 15 contiguous nucleotides any one of SEQ ID NOs: 73-80.

10. A dsRNA molecule according to claim 1, wherein the antisense region consists of any one of SEQ ID NOs: 73-80.

11. A dsRNA molecule according to claim 1,
wherein the sense region consists of a nucleotide sequence of nucleotides 1-19 of SEQ ID NO:49 and the antisense region consists of a nucleotide sequence of nucleotides 1-19 of SEQ ID NO:73,
wherein the sense region consists of a nucleotide sequence of nucleotides 1-19 of SEQ ID NO:50 and the antisense region consists of a nucleotide sequence of nucleotides 1-19 of SEQ ID NO:74,
wherein the sense region consists of a nucleotide sequence of nucleotides 1-19 of SEQ ID NO: 51 and the antisense region consists of a nucleotide sequence of nucleotides 1-19 of SEQ ID NO:75,
wherein the sense region consists of a nucleotide sequence of nucleotides 1-19 of SEQ ID NO:52 and the antisense region consists of a nucleotide sequence of nucleotides 1-19 of SEQ ID NO:76,
wherein the sense region consists of a nucleotide sequence of nucleotides 1-19 of SEQ ID NO:53 and the antisense region consists of a nucleotide sequence of nucleotides 1-19 of SEQ ID NO:77,
wherein the sense region consists of a nucleotide sequence of nucleotides 1-19 of SEQ ID NO:54 and the antisense region consists of a nucleotide sequence of nucleotides 1-19 of SEQ ID NO:78,
wherein the sense region consists of a nucleotide sequence of nucleotides 1-19 of SEQ ID NO:55 and the antisense region consists of a nucleotide sequence of nucleotides 1-19 of SEQ ID NO:79,
wherein the sense region consists of a nucleotide sequence of nucleotides 1-19 of SEQ ID NO:56 and the antisense region consists of a nucleotide sequence of nucleotides 1-19 of SEQ ID NO:80,
wherein the sense region consists of a nucleotide sequence of nucleotides 1-19 of SEQ ID NO:57 and the antisense region consists of a nucleotide sequence of nucleotides 1-19 of SEQ ID NO:81,
wherein the sense region consists of a nucleotide sequence of nucleotides 1-19 of SEQ ID NO:58 and the antisense region consists of a nucleotide sequence of nucleotides 1-19 of SEQ ID NO:82,
wherein the sense region consists of a nucleotide sequence of nucleotides 1-19 of SEQ ID NO:59 and the antisense region consists of a nucleotide sequence of nucleotides 1-19 of SEQ ID NO:83,
wherein the sense region consists of a nucleotide sequence of nucleotides 1-19 of SEQ ID NO: 60 and the antisense region consists of a nucleotide sequence of nucleotides 1-19 of SEQ ID NO:84,
wherein the sense region consists of a nucleotide sequence of nucleotides 1-19 of SEQ ID NO:61 and the antisense region consists of a nucleotide sequence of nucleotides 1-19 of SEQ ID NO:85,
wherein the sense region consists of a nucleotide sequence of nucleotides 1-19 of SEQ ID NO: 62 and the antisense region consists of a nucleotide sequence of nucleotides 1-19 of SEQ ID NO:86,
wherein the sense region consists of a nucleotide sequence of nucleotides 1-19 of SEQ ID NO: 63 and the antisense region consists of a nucleotide sequence of nucleotides 1-19 of SEQ ID NO:87,
wherein the sense region consists of a nucleotide sequence of nucleotides 1-19 of SEQ ID NO: 64 and the antisense region consists of a nucleotide sequence of nucleotides 1-19 of SEQ ID NO:88,
wherein the sense region consists of a nucleotide sequence of nucleotides 1-19 of SEQ ID NO: 65 and the antisense region consists of a nucleotide sequence of nucleotides 1-19 of SEQ ID NO:89,
wherein the sense region consists of a nucleotide sequence of nucleotides 1-19 of SEQ ID NO: 66 and the antisense region consists of a nucleotide sequence of nucleotides 1-19 of SEQ ID NO:90,
wherein the sense region consists of a nucleotide sequence of nucleotides 1-19 of SEQ ID NO: 67 and the antisense region consists of a nucleotide sequence of nucleotides 1-19 of SEQ ID NO:91,
wherein the sense region consists of a nucleotide sequence of nucleotides 1-19 of SEQ ID NO:68 and the antisense region consists of a nucleotide sequence of nucleotides 1-19 of SEQ ID NO:92,
wherein the sense region consists of a nucleotide sequence of nucleotides 1-19 of SEQ ID NO: 69 and the antisense region consists of a nucleotide sequence of nucleotides 1-19 of SEQ ID NO:93,
wherein the sense region consists of a nucleotide sequence of nucleotides 1-19 of SEQ ID NO:70 and the antisense region consists of a nucleotide sequence of nucleotides 1-19 of SEQ ID NO:94,
wherein the sense region consists of a nucleotide sequence of nucleotides 1-19 of SEQ ID NO: 71 and the antisense region consists of a nucleotide sequence of nucleotides 1-19 of SEQ ID NO:95, or
wherein the sense region consists of a nucleotide sequence of nucleotides 1-19 of SEQ ID NO: 72 and the antisense region consists of a nucleotide sequence of nucleotides 1-19 of SEQ ID NO:96.

12. A dsRNA molecule according to claim 1,
wherein the sense region consists of a nucleotide sequence of nucleotides 1-19 of SEQ ID NO:49 and the antisense region consists of a nucleotide sequence of nucelotides 1-19 SEQ ID 0:73,
wherein the sense region consists of a nucleotide sequence of nucleotides 1-19 of SEQ ID NO:50 and the antisense region consists of a nucleotide sequence of nucleotides 1-19 of SEQ ID NO:74,
wherein the sense region consists of a nucleotide sequence of nucleotides 1-19 of SEQ ID NO: 51 and the antisense region consists of a nucleotide sequence of SEQ ID NO:75,
wherein the sense region consists of a nucleotide sequence of nucleotides 1-19 of SEQ ID NO:52 and the antisense region consists of a nucleotide sequence of nucleotides 1-19 of SEQ ID NO:76,
wherein the sense region consists of a nucleotide sequence of nucleotides 1-19 of SEQ ID NO:53 and the antisense region consists of a nucleotide sequence of nucleotides 1-19 of SEQ ID NO:77,
wherein the sense region consists of a nucleotide sequence of nucleotides 1-19 of SEQ ID NO:54 and the antisense region consists of a nucleotide sequence of nucleotides 1-19 of SEQ ID NO:78,
wherein the sense region consists of a nucleotide sequence of nucleotides 1-19 of SEQ ID NO:55 and the antisense region consists of a nucleotide sequence of nucleotides 1-19 of SEQ ID NO:79, or
wherein the sense region consists of a nucleotide sequence of nucleotides 1-19 of SEQ ID NO:56 and the antisense region consists of a nucleotide sequence of nucleotides 1-19 of SEQ ID NO:80.

13. A dsRNA molecule according to claim 1, further comprising one or two overhang regions, each comprising five or fewer nucleotides.

14. A dsRNA molecule according to claim 13, wherein the one or two overhang regions comprise deoxythymidine (dT).

15. A dsRNA molecule according to claim 13, wherein the one or two overhang regions comprise dT-phosphorothioate-dT.

16. A dsRNA molecule according to claim 13, wherein the one or two overhang regions consist of dT-phosphorothioate-dT.

17. A dsRNA molecule according to claim 1, consisting of:
a first nucleotide strand comprising the sense region, wherein the first nucleotide strand consists of a nucleotide of SEQ ID NO:49 and a second nucleotide strand comprising the antisense strand, wherein the second nucleotide strand consists of a nucleotide of SEQ ID NO:73;
a first nucleotide strand comprising the sense region, wherein the first nucleotide strand consists of a nucleotide of SEQ ID NO:50 and a second nucleotide strand comprising the antisense strand, wherein the second nucleotide strand consists of a nucleotide of SEQ ID NO: 74;
a first nucleotide strand comprising the sense region, wherein the first nucleotide strand consists of a nucleotide of SEQ ID NO:51 and a second nucleotide strand comprising the antisense strand, wherein the second nucleotide strand consists of a nucleotide of SEQ ID NO:75;
a first nucleotide strand comprising the sense region, wherein the first nucleotide strand consists of a nucleotide of SEQ ID NO: 52 and a second nucleotide strand comprising the antisense strand, wherein the second nucleotide strand consists of a nucleotide of SEQ ID NO:76;
a first nucleotide strand comprising the sense region, wherein the first nucleotide strand consists of a nucleotide of SEQ ID NO:53 and a second nucleotide strand comprising the antisense strand, wherein the second nucleotide strand consists of a nucleotide of SEQ ID NO:77;
a first nucleotide strand comprising the sense region, wherein the first nucleotide strand consists of a nucleotide of SEQ ID NO: 54 and a second nucleotide strand comprising the antisense strand, wherein the second nucleotide strand consists of a nucleotide of SEQ ID NO:78;
a first nucleotide strand comprising the sense region, wherein the first nucleotide strand consists of a nucleotide of SEQ ID NO:55 and a second nucleotide strand comprising the antisense strand, wherein the second nucleotide strand consists of a nucleotide of SEQ ID NO: 79;
a first nucleotide strand comprising the sense region, wherein the first nucleotide strand consists of a nucleotide of SEQ ID NO:56 and a second nucleotide strand comprising the antisense strand, wherein the second nucleotide strand consists of a nucleotide of SEQ ID NO:80;
a first nucleotide strand comprising the sense region, wherein the first nucleotide strand consists of a nucleotide of SEQ ID NO:57 and a second nucleotide strand comprising the antisense strand, wherein the second nucleotide strand consists of a nucleotide of SEQ ID NO: 81;
a first nucleotide strand comprising the sense region, wherein the first nucleotide strand consists of a nucleotide of SEQ ID NO:58 and a second nucleotide strand comprising the antisense strand, wherein the second nucleotide strand consists of a nucleotide of SEQ ID NO:82;
a first nucleotide strand comprising the sense region, wherein the first nucleotide strand consists of a nucleotide of SEQ ID NO:59 and a second nucleotide
strand comprising the antisense strand, wherein the second nucleotide strand consists of a nucleotide of SEQ ID NO:83;
a first nucleotide strand comprising the sense region, wherein the first nucleotide strand consists of a nucleotide of SEQ ID NO: 60 and a second nucleotide strand comprising the antisense strand, wherein the second nucleotide strand consists of a nucleotide of SEQ ID NO: 84;
a first nucleotide strand comprising the sense region, wherein the first nucleotide strand consists of a nucleotide of SEQ ID NO:61 and a second nucleotide strand comprising the antisense strand, wherein the second nucleotide strand consists of a nucleotide of SEQ ID NO:85;
a first nucleotide strand comprising the sense region, wherein the first nucleotide strand consists of a nucleotide of SEQ ID NO: 62 and a second nucleotide strand comprising the antisense strand, wherein the second nucleotide strand consists of a nucleotide of SEQ ID NO:86;
a first nucleotide strand comprising the sense region, wherein the first nucleotide strand consists of a nucleotide of SEQ ID NO: 63 and a second nucleotide strand comprising the antisense strand, wherein the second nucleotide strand consists of a nucleotide of SEQ ID NO: 87;
a first nucleotide strand comprising the sense region, wherein the first nucleotide strand consists of a nucleotide of SEQ ID NO: 64 and a second nucleotide strand comprising the antisense strand, wherein the second nucleotide strand consists of a nucleotide of SEQ ID NO:88;
a first nucleotide strand comprising the sense region, wherein the first nucleotide strand consists of a nucleotide of SEQ ID NO: 65 and a second nucleotide strand comprising the antisense strand, wherein the second nucleotide strand consists of a nucleotide of SEQ ID NO:89;
a first nucleotide strand comprising the sense region, wherein the first nucleotide strand consists of a nucleotide of SEQ ID NO: 66 and a second nucleotide strand comprising the antisense strand, wherein the second nucleotide strand consists of a nucleotide of SEQ ID NO:90;
a first nucleotide strand comprising the sense region, wherein the first nucleotide strand consists of a nucleotide of SEQ ID NO: 67 and a second nucleotide strand comprising the antisense strand, wherein the second nucleotide strand consists of a nucleotide of SEQ ID NO: 91;
a first nucleotide strand comprising the sense region, wherein the first nucleotide strand consists of a nucleotide of SEQ ID NO: 68 and a second nucleotide strand comprising the antisense strand, wherein the second nucleotide strand consists of a nucleotide of SEQ ID NO:92;
a first nucleotide strand comprising the sense region, wherein the first nucleotide strand consists of a nucleotide of SEQ ID NO: 69 and a second nucleotide strand comprising the antisense strand, wherein the second nucleotide strand consists of a nucleotide of SEQ ID NO:93;
a first nucleotide strand comprising the sense region, wherein the first nucleotide strand consists of a nucleotide of SEQ ID NO: 70 and a second nucleotide strand comprising the antisense strand, wherein the second nucleotide strand consists of a nucleotide of SEQ ID NO: 94;
a first nucleotide strand comprising the sense region, wherein the first nucleotide strand consists of a nucleotide of SEQ ID NO:71 and a second nucleotide strand comprising the antisense strand, wherein the second nucleotide strand consists of a nucleotide of SEQ ID NO:95; or
a first nucleotide strand comprising the sense region, wherein the first nucleotide strand consists of a nucleotide of SEQ ID NO: 72 and a second nucleotide strand comprising the antisense strand, wherein the second nucleotide strand consists of a nucleotide of SEQ ID NO:96.

18. A dsRNA molecule according to claim 1,
a first nucleotide strand comprising the sense region, wherein the first nucleotide strand consists of a nucleotide of SEQ ID NO:49 and a second nucleotide strand comprising the antisense strand, wherein the second nucleotide strand consists of a nucleotide of SEQ ID NO:73;
a first nucleotide strand comprising the sense region, wherein the first nucleotide strand consists of a nucleotide of SEQ ID NO:50 and a second nucleotide strand comprising the antisense strand, wherein the second nucleotide strand consists of a nucleotide of SEQ ID NO: 74;
a first nucleotide strand comprising the sense region, wherein the first nucleotide strand consists of a nucleotide of SEQ ID NO: 51 and a second nucleotide strand comprising the antisense strand, wherein the second nucleotide strand consists of a nucleotide of SEQ ID NO:75;
a first nucleotide strand comprising the sense region, wherein the first nucleotide strand consists of a nucleotide of SEQ ID NO: 52 and a second nucleotide strand comprising the antisense strand, wherein the second nucleotide strand consists of a nucleotide of SEQ ID NO:76;
a first nucleotide strand comprising the sense region, wherein the first nucleotide strand consists of a nucleotide of SEQ ID NO:53 and a second nucleotide strand comprising the antisense strand, wherein the second nucleotide strand consists of a nucleotide of SEQ ID NO:77;
a first nucleotide strand comprising the sense region, wherein the first nucleotide strand consists of a nucleotide of SEQ ID NO: 54 and a second nucleotide strand comprising the antisense strand, wherein the second nucleotide strand consists of a nucleotide of SEQ ID NO:78;
a first nucleotide strand comprising the sense region, wherein the first nucleotide strand consists of a nucleotide of SEQ ID NO:55 and a second nucleotide strand comprising the antisense strand, wherein the second nucleotide strand consists of a nucleotide of SEQ ID NO:79; or
a first nucleotide strand comprising the sense region, wherein the first nucleotide strand consists of a nucleotide of SEQ ID NO:56 and a second nucleotide strand comprising the antisense strand, wherein the second nucleotide strand consists of a nucleotide of SEQ ID NO:80.

19. A method for inhibiting expression of beta catenin in a cell, comprising introducing into the cell a dsRNA molecule according to claim 1.

20. A pharmaceutically acceptable composition comprising a dsRNA molecule according to claim 1.

21-26. (canceled)
